(19) United States
(12) Patent Application Publication
(10) Pub. No.: US 2022/0211830 A1
(43) Pub. Date: Jul. 7, 2022

(54) CELL

(71) Applicant: AUTOLUS LIMITED, London (GB)

(72) Inventors: Martin Pulé, London (GB); Shaun Cordoba, London (GB); Simon Thomas, London (GB); Shimobi Onuoha, London (GB); James Sillibourne, London (GB); Farhaan Parekh, London (GB)

(21) Appl. No.: 17/601,684

(22) PCT Filed: Apr. 7, 2020
(86) PCT No.: PCT/GB2020/050908

§ 371 Date: Oct. 5, 2021

(30) Foreign Application Priority Data
Apr. 8, 2019 (GB) 1904971.7

(51) Int. Cl. A61K 39/00

(52) U.S. Cl. CPC A61K 39/0011; C07K 14/7051; A61K 2039/5158; A61K 2039/5156

(57) ABSTRACT

The present invention provides a cell which comprises; (i) a chimeric antigen receptor (CAR) or a transgenic T-cell receptor (TCR); and (ii) at least one polypeptide capable of co-localizing an MHC class I polypeptide or an MHC class II polypeptide with an intracellular signalling domain within the cell.

### FIELD OF THE INVENTION

The present invention relates to a cell which expresses a chimeric antigen receptor (CAR) or a T-cell receptor (TCR); and in particular to approaches to control immune rejection of such cells in a recipient.

### BACKGROUND TO THE INVENTION

After infusion, CAR T-cells engraft within the recipient and proliferate after encountering target bearing cells. CAR T-cells then persist and their population slowly contracts over time. CAR T-cell persistence can be determined in clinical studies by real-time PCR for the transgene in blood samples or by flow-cytometry for the CAR in blood samples and clinical researchers have found a correlation between persistence and sustained responses. This correlation is particularly pronounced in CD19 CAR therapy of B-Acute lymphoblastic leukaemia (ALL). Often in this setting, loss of CAR T-cell engraftment heralds relapse of the leukaemia.

CAR T-cells can result in activation of a cellular mediated immune response which can trigger rejection of the CAR T-cells. This is due to immunogenicity of the components engineered into the cell either through non-self proteins or through non-self sequences formed from junctions between self-proteins used to make receptors and other engineering components.

In many settings, CAR T-cells are generated from autologous T-cells. In this setting, allo-responses do not occur. However there are limitations to autologous CAR T cell production which include the need for a leukapheresis, a lag time to product release and poor quality of T cells for heavily treated patient donors.

In some circumstances, T-cells from an allogeneic donor are used. This can occur if for instance the patient has had an allogeneic haematopoietic stem cell transplant. In this case, harvested T-cells will be allogeneic. Allogeneic cells may be derived from a healthy donor or other source such as cord blood or induced pluripotent stem cells. Allogeneic CAR T-cells have advantages over autologous CAR T-cells; for instance, economies of scale in manufacture, better quality T-cells from a healthy donor. However, there are immunological barriers to allogeneic CAR T cells including rejection of the CAR T-cells by the recipient due to allo-responses, in such cases allogeneic T-cells can be rapidly rejected by the recipient, this limits their effectiveness. These allo-responses are caused by recognition of allo-HLA by recipient T-cells (major histocompatibility rejection or by recognition of alloreactive peptides presented by HLA on the CAR T-cells (minor histocompatibility rejection).

There are two classes of HLA: class I and class II. Solutions have been proposed to prevent allo-rejection by MHC class I MHC.

For example, one method for preventing rejection by cellular immune responses is by genomic editing tools such as engineered zinc finger nucleases, TALENs, CrispR/Casp9, MegaTALs and meganucleases. Using such tools elements of peptide/HLA presentation can be disrupted. The most direct way of doing this is by disrupting HLA expression. This can be achieved by disruption of the HLA locus or alternatively by disruption of the beta-2 microglobulin (B2M) locus (which then stops MHC class I expression). Other approaches include disrupting of components of peptide presentation.

Another method of preventing cellular mediated immune rejection relies on protein based approaches to disrupt HLA expression. For instance, an antibody single-chain variable fragment which recognizes B2M and which has a Golgi/ER retention signal at its carboxy terminus can result in down-regulation of HLA since B2M is retained within the ER/Golgi complex. Other approaches include using viral proteins which have evolved to disrupt HLA expression and function.

The main limitation of all these approaches is they rely on or result in surface down-regulation of class I which in turn triggers rejection by NK cells. Hence these approaches solve one problem of alpha/beta T-cell mediated cellular rejection but cause another type of cellular immune-rejection, namely that by NK cells.

However rejection can also occur via HLA class II. Immune effector cells upregulate HLA class II upon activation, and therefore some of the allo-responses can be targeted at HLA class II. Activated T cells synthesise and express MHC class II molecules at their cell surface.

There is therefore a need for alternative approaches to reduce HLA class II cellular mediated immune rejection of engineered cells, in particular engineered immune cells expressing a CAR or an engineered-TCR.

### SUMMARY OF ASPECTS OF THE INVENTION

The present inventors have found that it is possible to couple the binding of an MHC class I polypeptide or MHC class II polypeptide on a first cell to a second cell to induce—directly or indirectly—signalling in the first cell. Notably, the present MHC class I and MHC class II signalling systems are capable of presenting the same range of peptides as a corresponding endogenous MHC class I or MHC class II molecules. As such, any peptide which is naturally presented by MHC class I or MHC class II is presented by the MHC class I or MHC class II of the present invention. This includes any xenogeneic peptides that may be immunogenic. In an allogeneic setting, this may also include minor histocompatibility antigens. Thus an MHC class I or MHC class II as defined herein will interact with any endogenous, reactive T-cells present in the recipient of an engineered cell of the present invention through recognition of peptide/MHC complexes. The reactive T-cell can thus be depleted by activation of cytotoxic-mediated cell killing by the cell of the present invention. Hence, an immune response against the cell of the present invention can be reduced.

Thus, in a first aspect, the present invention provides a cell which comprises:

(i) a chimeric antigen receptor (CAR) or a transgenic T-cell receptor (TCR); and

(ii) at least one polypeptide capable of co-localizing an MHC class I polypeptide or an MHC class II polypeptide with an intracellular signalling domain within the cell.

The cell may comprise:

(i) a chimeric antigen receptor (CAR) or a transgenic T-cell receptor (TCR); and

(ii) an engineered polypeptide which comprises the ectodomain from an MHC class I polypeptide or MHC class II polypeptide linked to an intracellular signalling domain.

The cell may comprise:

(i) a chimeric antigen receptor (CAR) or a transgenic T-cell receptor (TCR); and

(ii) an engineered polypeptide which comprises an MHC class I polypeptide or MHC class II polypeptide linked to linked to a component of the CD3/TCR complex.

The component of the CD3/TCR complex may, for example, be selected from CD3-zeta, CD3-epsilon, CD3-gamma and CD3-delta.

The cell may comprise:

(i) a chimeric antigen receptor (CAR) or a transgenic T-cell receptor (TCR); and

(ii) an engineered polypeptide which comprises a binding domain which is binds to an MHC class I polypeptide or an MHC class II polypeptide linked to an intracellular signalling domain.

The cell may comprise:

(i) a chimeric antigen receptor (CAR) or a transgenic T-cell receptor (TCR); and

(ii) an engineered polypeptide which comprises CD79α or CD79β linked to an intracellular signalling domain.

The cell may comprise:

(i) a chimeric antigen receptor (CAR) or a transgenic T-cell receptor (TCR); and

(ii) an engineered polypeptide which comprises the MHC class II-binding domain of CD4 linked to an intracellular signalling domain.

The MHC class II-binding domain of CD4 may comprise one or more mutations to increase its binding affinity for the β2 region of MHC class II. For example, the MHC class II-binding domain of CD4 may comprise substitution mutations Gln40Tyr and/or Thr45Trp with reference to SEQ ID No. 47.

The cell may comprise:

(i) a chimeric antigen receptor (CAR) or a transgenic T-cell receptor (TCR); and

(ii) an engineered polypeptide which comprises the MHC class I-binding domain of CD8 linked to an intracellular signalling domain.

The cell may comprise:

(i) a chimeric antigen receptor (CAR) or a transgenic T-cell receptor (TCR); and expresses

(ii) a bispecific polypeptide which comprises;
(a) a first binding domain which is binds to an MHC class I polypeptide or an MHC class II polypeptide; and
(b) a second binding domain which binds to a polypeptide comprising an intracellular signalling domain or a component of the CD3 complex.

The bispecific polypeptide may be membrane-tethered.

In a second aspect, the present invention provides a nucleic acid construct which comprises:

(i) a first nucleic acid sequence which encodes a chimeric antigen receptor (CAR) or a transgenic TCR; and

(ii) a second nucleic acid sequence which encodes an engineered polypeptide or a bispecific polypeptide as defined above.

The first and second nucleic acid sequences may be separated by a co-expression site.

In a third aspect, the present invention provides a kit of nucleic acid sequences comprising:

(i) a first nucleic acid sequence which encodes a chimeric antigen receptor (CAR) or a transgenic TCR; and

(ii) a second nucleic acid sequence which encodes an engineered polypeptide or a bispecific polypeptide as defined above.

In a fourth aspect, the present invention provides a vector which comprises a nucleic acid construct according to the second aspect of the invention.

In a fifth aspect, the present invention provides a kit of vectors which comprises:

(i) a first vector which comprises a first nucleic acid sequence which encodes a chimeric antigen receptor (CAR) or a transgenic TCR; and

(ii) a second vector which comprises a second nucleic acid sequence which encodes an engineered polypeptide or a bispecific polypeptide as defined above.

In a sixth aspect, the present invention provides a pharmaceutical composition which comprises a plurality of cells according to the first aspect of the invention.

In a seventh aspect, the present invention provides a pharmaceutical composition according to sixth aspect of the invention for use in treating a disease.

In an eighth aspect, the invention provides a method for treating a disease, which comprises the step of administering a pharmaceutical composition according to the sixth aspect of the invention to a subject.

In a ninth aspect, there is provided the use of a cell according to the first aspect of the invention in the manufacture of a medicament for the treatment of a disease.

The disease may be cancer.

In a tenth aspect, there is provided a method for making a cell according to the first aspect of the invention, which comprises the step of introducing: a nucleic acid construct according to the second aspect of the invention; a kit of nucleic acid sequences according to the third aspect of the invention; a vector according to the fourth aspect of the invention; or a kit of vectors according to the fifth aspect of the invention, into a cell ex vivo.

In an eleventh aspect, there is provided a method for depleting alloreactive immune cells from a population of immune cells, which comprises the step of contacting the population of immune cells with a plurality of cells which express an engineered polypeptide or a bispecific polypeptide as defined above.

In a twelfth aspect, there is provided a method for treating or preventing graft rejection following allotransplantation, which comprises the step of administering a plurality of cells derived from the donor subject to the recipient subject for the allotransplant, wherein the plurality of cells express an engineered polypeptide or a bispecific polypeptide as defined above.

In a thirteenth aspect, there is provided a method for treating or preventing graft versus host disease (GVHD) associated with allotransplantation, which comprises the step of contacting the allotransplant with administering a plurality of cells which express an engineered polypeptide or a bispecific polypeptide as defined above.

The allotransplantation may comprise adoptive transfer of allogeneic immune cells.

In a fourteenth aspect, there is provided an allotransplant which has been depleted of alloreactive immune cells by a method according to the thirteenth aspect of the invention.

### DESCRIPTION OF THE FIGURES

FIG. 1—(a) A naturally occurring MHC class II molecular complex which is composed of an α chain and a β chain, for example HLA-DRα and HLA-DRβ and presents a peptide; (b) MHC class II molecule comprising an α and a β chain in association with CD79, which comprises CD79α and CD79β which may both contain signalling domains; (c) an engineered MHC class II molecule which comprises an α chain and a β chain wherein the α chain comprises a signalling domain.

FIG. 2—MHC class I and TCR

(a) MHC class I molecules are heterodimers that consist of two polypeptide chains, α and β2-microglobulin (B2M); (b) The TCR complex which is composed of TCRalpha/beta chains surrounded by CD3 elements

FIG. 3—Different MHClα/TCR fusion constructs

(a) MHClα-CD3z construct: The MHC class I alpha chain is fused in frame to a TM domain and CD3-zeta endodomain; (b) Ab-CD3z construct: An antibody or antibody-like binder specific to MHC class I alpha chain is fused to a TM domain and CD3-zeta endodomain; (c) Fusion between MHClα and CD3/TCR: As an example, a fusion between MHC class I alpha chain via a flexible linker to CD3 Epsilon is shown; (d) MHClα-TCR BiTE construct: a scFv which recognizes MHC class I alpha chain is fused with a linker to a second scFv which recognizes the CD3/TCR complex. This is then anchored to the membrane via a transmembrane domain.

FIG. 4—MHC class II and TCR

(a) MHC class II molecules are heterodimers that consist an α chain and a β chain; (b) The TCR complex which is composed of TCRalpha/beta chains surrounded by CD3 elements

FIG. 5—Different MHCII/TCR fusion constructs

(a) MHCII-CD3z construct: The MHC class II α or β chain is fused to a TM domain and CD3-zeta endodomain; (b) Ab-CD3z construct: An antibody or antibody-like binder specific to MHC class II α or β chain is fused to a TM domain and CD3-zeta endodomain; (c) Fusion between MHCII and CD3/TCR: MHC class I α or β chain is fused via a flexible linker to a component of the TCR/CD3 complex. For example, CD3 Epsilon is shown; (d) MHCII-TCR BITE construct: a scFv which recognizes MHC class II α or β chain is fused with a linker to a second scFv which recognizes the CD3/TCR complex. This is then anchored to the membrane via a transmembrane domain.

FIG. 6—CD4/CD8 fusion molecules

CD4 and CD8 are TCR co-receptors. The extracellular domain of CD4 binds to the β2 region of MHC class II; whereas the extracellular domain of CD8 binds the α3 portion of the Class I MHC molecule. (a) CD4-CD3z construct: the MHC class II-binding domain of CD4 is fused to a TM domain and CD3-zeta endodomain; (b) CD8-CD3z construct: the MHC class I-binding domain of CD8 is fused to a TM domain and CD3-zeta endodomain

FIG. 7—Upregulation of MHC II on Jurkat cells upon transduction with CIITA

Jurkat cells were co-transduced with the chimeric receptors and eGFP or eGFP and CIITA. The cells were stained with anti-DRα antibody and antibodies to the transduction markers. Overexpression of CIITA induces cell surface expression of DRα.

FIG. 8—Schematic diagram illustrating cytotoxicity assays using CD+ target cells expressing HA1.7α/βTCR and effector cells with MHC II/CD3ζ chimeric receptors.

Target cells are CD4+ to enable recognition of MHC II molecules and are transduced to express the HA1.7 α/βTCR and HA immunodominant peptide. Cytotoxic T-cells are transduced with MHC II/CD3z chimeric receptors and challenged with the target cells described above to determine their ability to cytolyse them.

### DETAILED DESCRIPTION OF THE INVENTION

Co-Localizing an MHC Class I or MHC Class II Polypeptide with an Intracellular Signalling Domain

The present invention provides a cell which comprises; (i) a chimeric antigen receptor (CAR) or a transgenic T-cell receptor (TCR); and (ii) at least one polypeptide capable of co-localizing an MHC class I polypeptide or MHC class II polypeptide with an intracellular signalling domain within the cell.

The cell may comprise an engineered polypeptide which comprises the ectodomain from an MHC class I polypeptide or MHC class II polypeptide linked to an intracellular signalling domain.

HLA Class I

MHC class I molecules are a class of major histocompatibility complex (MHC) molecules which are found on the cell surface of all nucleated cells and platelets in the bodies of jawed vertebrates. The function of MHC class I molecules is to display peptide fragments of proteins from the cytosol to cytotoxic T cells, thereby initiating a response from the immune system against non-self antigen displayed by MHC class I protein.

In humans, the MHC class I molecules are heterodimers that consist of two polypeptide chains, an α polypeptide and β2-microglobulin (b2m). The two chains are linked non-covalently via interaction of b2m and the α3 domain. The α chain is polymorphic and encoded by a human leukocyte antigen gene complex (HLA). The b2m subunit is not polymorphic and encoded by the Beta-e macroglobulin gene. HLA gene. HLAs corresponding to MHC class I are HLA-A, HLA-B, HLA-C, HLA-E, HLA-F, HLA-G.

HLA-A, HLA-B and HLA-C are typically very polymorphic whilst HLA-E, HLA-F, HLA-G are less polymorphic.

In one embodiment, the at least one polypeptide capable of co-localizing the MHC class I polypeptide with the intracellular signalling domain is an MHC class I α polypeptide. Suitably, the at least one polypeptide capable of co-localizing the MHC class I polypeptide with the intracellular signalling domain may be an MHC class I α polypeptide comprising an intracellular signalling domain.

In one embodiment, the at least one polypeptide capable of co-localizing the MHC class I polypeptide with the intracellular signalling domain comprises an ectodomain from HLA-A and an intracellular signalling domain.

In one embodiment, the at least one polypeptide capable of co-localizing the MHC class I polypeptide with the intracellular signalling domain comprises an ectodomain from HLA-B and an intracellular signalling domain.

In one embodiment, the at least one polypeptide capable of co-localizing the MHC class I polypeptide with the intracellular signalling domain comprises an ectodomain from HLA-C and an intracellular signalling domain.

The most common haplotypes vary between populations. Accordingly a cell according to the present invention may be designed for a certain population with specific common haplotypes. Polypeptides for any haplotype or any combination of haplotypes may be used in the present invention. Exemplary haplotypes for use according to the present invention include any of those recited in the table below:

TABLE 1
Exemplary common haplotypes.

| HLA-A | HLA-B | HLA-C |
|---|---|---|
| HLA-A02 | HLA-B08 | HLA-C01 |
| HLA-A03 | HLA-B07 | |
| HLA-A01 | HLA-B44 | |
| HLA-A29 | HLA-B15 | |
| HLA-A30 | HLA-B35 | |

An illustrative amino acid sequence of HLA class I—HLA-A is HLA-A01 as shown as SEQ ID NO: 28:

SEQ ID NO: 28
MAVMAPRTLLLLLSGALALTQTWAGSHSMRYFFTSVSRPGRGEPRFIAVG
YVDDTQFVRFDSDAASQKMEPRAPWIEQEGPEYWDQETRNMKAHSQTDRA
NLGTLRGYYNQSEDGSHTIQIMYGCDVGPDGRFLRGYRQDAYDGKDYIAL
NEDLRSWTAADMAAQITKRKWEAVHAAEQRRVYLEGRCVDGLRRYLENGK
ETLQRTDPPKTHMTHHPISDHEATLRCWALGFYPAEITLTWQRDGEDQTQ
DTELVETRPAGDGTFQKWAAVVVPSGEEQRYTCHVQHEGLPKPLTLRWEL
SSQPTIP [symbol] [symbol] MWRRKSSDRKGGSYTQAA
SSDSAQGSDVSLTACKV

Ectodomain=unformatted text

Bold/underline=transmembrane

Italics=endodomain

Suitably, the at least one polypeptide capable of co-localizing an MHC class I polypeptide with an intracellular signalling domain within the cell may comprise an ectodomain from HLA-A. Suitably, the at least one polypeptide capable of co-localizing a MHC class I polypeptide with an intracellular signalling domain within the cell may comprise an ectodomain from HLA-A01. Suitably, the at least one polypeptide capable of co-localizing a MHC class I polypeptide with an intracellular signalling domain within the cell may comprise an ectodomain from HLA-A01 as set forth in SEQ ID NO: 28 (such as from about amino acid 1 to about amino acid 307 of SEQ ID NO: 28) or a variant thereof having at least 80% sequence identity. It will be understood that the amino acid sequence (such as the sequence from amino acid 1 to about amino acid 307 set forth above) may comprise a signal peptide.

An HLA-A sequence for use in the present invention may comprise the sequence shown as SEQ ID NO: 28 or a variant thereof having at least 80% sequence identity. The variant maintains ability to assemble and facilitate productive peptide presentation by the MHC class I complex.

The variant of SEQ ID NO: 28 may have at least 80, 85, 90, 95, 98 or 99% sequence identity, provided that the variant maintains ability to assemble and facilitate productive peptide presentation by the MHC class I complex.

An illustrative amino acid sequence of HLA class I—HLA-A is HLA-A02 as shown as SEQ ID NO: 29:

SEQ ID NO: 29
MAVMAPRTLVLLLSGALALTQTWAGSHSMRYFFTSVSRPGRGEPRFIAVG
YVDDTQFVRFDSDAASQRMEPRAPWIEQEGPEYWDGETRKVKAHSQTHRV
DLGTLRGYYNQSEAGSHTVQRMYGCDVGSDWRFLRGYHQYAYDGKDYIAL
KEDLRSWTAADMAAQTTKHKWEAAHVAEQLRAYLEGTCVEWLRRYLENGK
ETLQRTDAPKTHMTHHAVSDHEATLRCWALSFYPAEITLTWQRDGEDQTQ
DTELVETRPAGDGTFQKWAAVVVPSGQEQRYTCHVQHEGLPKPLTLRWEP
SSQPTIP [symbol] [symbol] MWRRKSSDRKGGSYSQAA
SSDSAQGSDVSLTACKV

Ectodomain=unformatted text

Bold/underline=transmembrane

Italics=endodomain

Suitably, the at least one polypeptide capable of co-localizing an MHC class I polypeptide with an intracellular signalling domain within the cell may comprise an ectodomain from HLA-A. Suitably, the at least one polypeptide capable of co-localizing a MHC class I polypeptide with an intracellular signalling domain within the cell may comprise an ectodomain from HLA-A02. Suitably, the at least one polypeptide capable of co-localizing a MHC class I polypeptide with an intracellular signalling domain within the cell may comprise an ectodomain from HLA-A02 as set forth in SEQ ID NO: 29 (such as from about amino acid 1 to about amino acid 307 of SEQ ID NO: 29) or a variant thereof having at least 80% sequence identity. It will be understood that the amino acid sequence (such as the sequence from amino acid 1 to about amino acid 307 set forth above) may comprise a signal peptide.

An HLA-A sequence for use in the present invention may comprise the sequence shown as SEQ ID NO: 29 or a variant thereof having at least 80% sequence identity. The variant maintains ability to assemble and facilitate productive peptide presentation by the MHC class I complex.

The variant of SEQ ID NO: 29 may have at least 80, 85, 90, 95, 98 or 99% sequence identity, provided that the variant maintains ability to assemble and facilitate productive peptide presentation by the MHC class I complex.

An illustrative amino acid sequence of HLA class I—HLA-A is HLA-A-A03 as shown as SEQ ID NO: 30:

SEQ ID NO: 30
MAVMAPRTLLLLLSGALALTQTWAGSHSMRYFFTSVSRPGRGEPRFIAVG
YVDDTQFVRFDSDAASQRMEPRAPWIEQEGPEYWDQETRNVKAQSQTDRV
DLGTLRGYYNQSEAGSHTIQIMYGCDVGSDGRFLRGYRQDAYDGKDYIAL
NEDLRSWTAADMAAQITKRKWEAAHEAEQLRAYLDGTCVEWLRRYLENGK
ETLQRTDPPKTHMTHHPISDHEATLRCWALGFYPAEITLTWQRDGEDQTQ
DTELVETRPAGDGTFQKWAAVVVPSGEEQRYTCHVQHEGLPKPLTLRWEL
SSQPTIP [symbol] [symbol] MWRRKSSDRKGGSYTQAA
SSDSAQGSDVSLTACKV

Ectodomain=unformatted text

Bold/underline=transmembrane

Italics=endodomain

Suitably, the at least one polypeptide capable of co-localizing an MHC class I polypeptide with an intracellular signalling domain within the cell may comprise an ectodomain from HLA-A. Suitably, the at least one polypeptide capable of co-localizing a MHC class I polypeptide with an intracellular signalling domain within the cell may comprise an ectodomain from HLA-A03. Suitably, the at least one polypeptide capable of co-localizing a MHC class I polypeptide with an intracellular signalling domain within the cell may comprise an ectodomain from HLA-A03 as set forth in SEQ ID NO: 30 (such as from about amino acid 1 to about amino acid 307 of SEQ ID NO: 30) or a variant thereof having at least 80% sequence identity. It will be understood that the amino acid sequence (such as the sequence from amino acid 1 to about amino acid 307 set forth above) may comprise a signal peptide.

An HLA-A sequence for use in the present invention may comprise the sequence shown as SEQ ID NO: 30 or a variant thereof having at least 80% sequence identity. The variant maintains ability to assemble and facilitate productive peptide presentation by the MHC class I complex.

The variant of SEQ ID NO: 30 may have at least 80, 85, 90, 95, 98 or 99% sequence identity, provided that the variant maintains ability to assemble and facilitate productive peptide presentation by the MHC class I complex.

An illustrative amino acid sequence of HLA class I—HLA-B is HLA-B07 as shown as SEQ ID NO: 31:

SEQ ID NO: 31
MLVMAPRTVLLLLSAALALTETWAGSHSMRYFYTSVSRPGRGEPRFISVG
YVDDTQFVRFDSDAASPREEPRAPWIEQEGPEYWDRNTQIYKAQAQTDRE
SLRNLRGYYNQSEAGSHTLQSMYGCDVGPDGRLLRGHDQYAYDGKDYIAL
NEDLRSWTAADTAAQITQRKWEAAREAEQRRAYLEGECVEWLRRYLENGK
DKLERADPPKTHVTHHPISDHEATLRCWALGFYPAEITLTWQRDGEDQTQ
DTELVETRPAGDRTFQKWAAVVVPSGEEQRYTCHVQHEGLPKPLTLRWEP
SSQSTVP [symbol] [symbol] MCRRKSSGGKGGSYSQAACS
DSAQGSDVSLTA

Ectodomain=unformatted text

Bold/underline=transmembrane

Italics=endodomain

Suitably, the at least one polypeptide capable of co-localizing an MHC class I polypeptide with an intracellular signalling domain within the cell may comprise an ectodomain from HLA-B. Suitably, the at least one polypeptide capable of co-localizing a MHC class I polypeptide with an intracellular signalling domain within the cell may comprise an ectodomain from HLA-807. Suitably, the at least one polypeptide capable of co-localizing a MHC class I polypeptide with an intracellular signalling domain within the cell may comprise an ectodomain from HLA-B07 as set forth in SEQ ID NO: 31 (such as from about amino acid 1 to about amino acid 307 of SEQ ID NO: 31) or a variant thereof having at least 80% sequence identity. It will be understood that the amino acid sequence (such as the sequence from amino acid 1 to about amino acid 307 set forth above) may comprise a signal peptide.

An HLA-B07 sequence for use in the present invention may comprise the sequence shown as SEQ ID NO: 31 or a variant thereof having at least 80% sequence identity. The variant maintains ability to assemble and facilitate productive peptide presentation by the MHC class I complex.

The variant of SEQ ID NO: 31 may have at least 80, 85, 90, 95, 98 or 99% sequence identity, provided that the variant maintains ability to assemble and facilitate productive peptide presentation by the MHC class I complex.

An illustrative amino acid sequence of HLA class I—HLA-B is HLA-B08 as shown as SEQ ID NO: 32:

SEQ ID NO: 32
MLVMAPRTVLLLLSAALALTETWAGSHSMRYFDTAMSRPGRGEPRFISVG
YVDDTQFVRFDSDAASPREEPRAPWIEQEGPEYWDRNTQIFKTNTQTDRE
SLRNLRGYYNQSEAGSHTLQSMYGCDVGPDGRLLRGHNQYAYDGKDYIAL
NEDLRSWTAADTAAQITQRKWEAARVAEQDRAYLEGTCVEWLRRYLENGK
DTLERADPPKTHVTHHPISDHEATLRCWALGFYPAEITLTWQRDGEDQTQ
DTELVETRPAGDRTFQKWAAVVVPSGEEQRYTCHVQHEGLPKPLTLRWEP
SSQSTVP [symbol] [symbol] MCRRKSSGGKGGSYSQAAC
SDSAQGSDVSLTA

Ectodomain=unformatted text

Bold/underline=transmembrane

Italics=endodomain

Suitably, the at least one polypeptide capable of co-localizing an MHC class I polypeptide with an intracellular signalling domain within the cell may comprise an ectodomain from HLA-B. Suitably, the at least one polypeptide capable of co-localizing a MHC class I polypeptide with an intracellular signalling domain within the cell may comprise an ectodomain from HLA-B08. Suitably, the at least one polypeptide capable of co-localizing a MHC class I polypeptide with an intracellular signalling domain within the cell may comprise an ectodomain from HLA-B08 as set forth in SEQ ID NO: 32 (such as from about amino acid 1 to about amino acid 307 of SEQ ID NO: 32) or a variant thereof having at least 80% sequence identity. It will be understood that the amino acid sequence (such as the sequence from amino acid 1 to about amino acid 307 set forth above) may comprise a signal peptide.

An HLA-B08 sequence for use in the present invention may comprise the sequence shown as SEQ ID NO: 32 or a variant thereof having at least 80% sequence identity. The variant maintains ability to assemble and facilitate productive peptide presentation by the MHC class I complex.

The variant of SEQ ID NO: 32 may have at least 80, 85, 90, 95, 98 or 99% sequence identity, provided that the variant maintains ability to assemble and facilitate productive peptide presentation by the MHC class I complex.

An illustrative amino acid sequence of HLA class I—HLA-B is HLA-B44 as shown as SEQ ID NO: 33:

SEQ ID NO: 33
MRVTAPRILLLLLWGAVALTETWAGSHSMRYFYTAMSRPGRGEPRFITVG
YVDDTLEVREDSDATSPRKEPRAPWIEQEGPEYWDRETQISKTNTQTYRE
NLRTALRYYNQSEAGSHIIQRMYGCDVGPDGRLLRGYDQDAYDGKDYIAL
NEDLSSWTAADTAAQITQRKWEAARVAEQDRAYLEGLCVESLRRYLENGK
ETLQRADPPKTHVTHHPISDHEVTLRCWALGFYPAEITLTWQRDGEDQTQ
DTELVETRPAGDRTFQKWAAVVVPSGEEQRYTCHVQHEGLPKPLTLRWEP
SSQSTVP [symbol] [symbol] MCRRKSSGGKGGSYSQAA
CSDSAQGSDVSLTA

Ectodomain=unformatted text

Bold/underline=transmembrane

Italics=endodomain

Suitably, the at least one polypeptide capable of co-localizing an MHC class I polypeptide with an intracellular signalling domain within the cell may comprise an ectodomain from HLA-B. Suitably, the at least one polypeptide capable of co-localizing a MHC class I polypeptide with an intracellular signalling domain within the cell may comprise an ectodomain from HLA-B44. Suitably, the at least one polypeptide capable of co-localizing a MHC class I polypeptide with an intracellular signalling domain within the cell may comprise an ectodomain from HLA-B44 as set forth in SEQ ID NO: 33 (such as from about amino acid 1 to about amino acid 307 of SEQ ID NO: 33) or a variant thereof having at least 80% sequence identity. It will be understood that the amino acid sequence (such as the sequence from amino acid 1 to about amino acid 307 set forth above) may comprise a signal peptide.

An HLA-B44 sequence for use in the present invention may comprise the sequence shown as SEQ ID NO: 33 or a variant thereof having at least 80% sequence identity. The variant maintains ability to assemble and facilitate productive peptide presentation by the MHC class I complex.

The variant of SEQ ID NO: 33 may have at least 80, 85, 90, 95, 98 or 99% sequence identity, provided that the variant maintains ability to assemble and facilitate productive peptide presentation by the MHC class I complex.

An illustrative amino acid sequence of HLA class I—HLA-C is HLA-001 as shown as SEQ ID NO: 34:

SEQ ID NO: 34
MRVMAPRTLILLLSGALALTETWACSHSMKYFFTSVSRPGRGEPRFISVG
YVDDTQFVRFDSDAASPRGEPRAPWVEQEGPEYWDRETQKYKRQAQTDRV
SLRNLRGYYNQSEAGSHTLQWMCGCDLGPDGRLLRGYDQYAYDGKDYIAL
NEDLRSWTAADTAAQITQRKWEAAREAEQRRAYLEGTCVEWLRRYLENGK
ETLQRAEHPKTHVTHHPVSDHEATLRCWALGFYPAEITLTWQWDGEDQTQ
DTELVETRPAGDGTFQKWAAVMVPSGEEQRYTCHVQHEGLPEPLTLRWEP
SSQPTIP [symbol] [symbol] MCRRKSSGGKGGSCSQAAS
SNSAQGSDESLIACKA

Ectodomain=unformatted text

Bold/underline=transmembrane

Italics=endodomain

Suitably, the at least one polypeptide capable of co-localizing an MHC class I polypeptide with an intracellular signalling domain within the cell may comprise an ectodomain from HLA-C. Suitably, the at least one polypeptide capable of co-localizing a MHC class I polypeptide with an intracellular signalling domain within the cell may comprise an ectodomain from HLA-C-001.

Suitably, the at least one polypeptide capable of co-localizing a MHC class I polypeptide with an intracellular signalling domain within the cell may comprise an ectodomain from HLA-001 as set forth in SEQ ID NO: 34 (such as from about amino acid 1 to about amino acid 307 of SEQ ID NO: 34) or a variant thereof having at least 80% sequence identity. It will be understood that the amino acid sequence (such as the sequence from amino acid 1 to about amino acid 307 set forth above) may comprise a signal peptide.

An HLA-001 sequence for use in the present invention may comprise the sequence shown as SEQ ID NO: 34 or a variant thereof having at least 80% sequence identity. The variant maintains ability to assemble and facilitate productive peptide presentation by the MHC class I complex.

The variant of SEQ ID NO: 34 may have at least 80, 85, 90, 95, 98 or 99% sequence identity, provided that the variant maintains ability to assemble and facilitate productive peptide presentation by the MHC class I complex.

Engineered MHC Class I Polypeptide

In some embodiments, the at least one polypeptide capable of co-localizing the MHC class I polypeptide with the intracellular signalling domain comprises an ectodomain from HLA-A, HLA-B or HLA-C and an intracellular signalling domain.

In one embodiment the HLA-A is HLA-A01. Suitably, the at least one polypeptide may comprise an ectodomain from HLA-A01 and an intracellular signalling domain.

In one embodiment the HLA-A is HLA-A02. Suitably, the at least one polypeptide may comprise an ectodomain from HLA-A02 and an intracellular signalling domain.

In one embodiment the HLA-A is HLA-A03. Suitably, the at least one polypeptide may comprise an ectodomain from HLA-A03 and an intracellular signalling domain.

In one embodiment the HLA-A is HLA-B07. Suitably, the at least one polypeptide may comprise an ectodomain from HLA-B07 and an intracellular signalling domain.

In one embodiment the HLA-A is HLA-B08. Suitably, the at least one polypeptide may comprise an ectodomain from HLA-B08 and an intracellular signalling domain.

In one embodiment the HLA-A is HLA-B44. Suitably, the at least one polypeptide may comprise an ectodomain from HLA-B44 and an intracellular signalling domain.

In one embodiment the HLA-A is HLA-001. Suitably, the at least one polypeptide may comprise an ectodomain from HLA-001 and an intracellular signalling domain.

Such an engineered polypeptide comprises an MHC class I ectodomain and is capable of facilitating productive peptide presentation by the MHC class I complex on the cell surface. In addition, the polypeptide further comprises an intracellular signalling domain that is capable of transmitting an activating signal following binding of a TCR to the peptide/MHC complex which comprises the engineered polypeptide. The intracellular signalling domain may be an intracellular signalling domain as described herein.

A T-cell expressing such a polypeptide will selectively deplete any reactive T-cell which recognizes the selected peptide/MHC complex. In this way, selective immunosuppression against a known antigen can be executed by depletion of cognate T-cells. This advantageously enables a cell expressing the present MHC class I complexes to deplete endogenous, reactive T cells which recognise any peptide/MHC complex which is presented by the cell of the present invention.

In some embodiments the at least one polypeptide capable of co-localizing the MHC class I polypeptide with the intracellular signalling domain comprises an ectodomain from HLA-A, HLA-B or HLA-C, a transmembrane domain and an intracellular signalling domain. Suitably, the polypeptide may comprise a transmembrane domain located between the HLA ectodomain and the endodomain comprising an intracellular signalling domain.

The transmembrane domain may be any peptide domain that is capable of inserting into and spanning the cell membrane. A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (U.S. Pat. No. 7,052,906 B1 describes synthetic transmembrane components). For example, the transmembrane domain may comprise a hydrophobic alpha helix. The transmembrane domain may be derived from CD8alpha or CD28.

An illustrative polypeptide for use in the present invention is shown as SEQ ID NO: 35

(HLA-A01-CAR)
SEQ ID NO: 35
MAVMAPRTLLLLLSGALALTQTWAGSHSMRYFFTSVSRPGRGEPRFIAVG
YVDDTQFVRFDSDAASQKMEPRAPWIEQEGPEYWDQETRNMKAHSQTDRA
NLGTLRGYYNQSEDGSHTIQIMYGCDVGPDGRFLRGYRQDAYDGKDYIAL
NEDLRSWTAADMAAQITKRKWEAVHAAEQRRVYLEGRCVDGLRRYLENGK
ETLQRTDPPKTHMTHHPISDHEATLRCWALGFYPAEITLTWQRDGEDQTQ
DTELVETRPAGDGTFQKWAAVVVPSGEEQRYTCHVQHEGLPKPLTLRWEL
SSQPTIP [symbol] [symbol] KFSRSADAPAYQQGQNQL
YNELNLGRREEYDVLDKRRGRDPEMGGKPQRRKNPQEGLYNELQKDKMAE
AYSEIGMKGERRRGKGHDGLYQGLSTATKDTYDALHMQALPPR

Ectodomain=unformatted text

Bold/underlined=transmembrane

Italics=endodomain

An illustrative polypeptide for use in the present invention is shown as SEQ ID NO: 36

(HLA-A02-CAR)
SEQ ID NO: 36
MAVMAPRTLVLLLSGALALTQTWAGSHSMRYFFTSVSRPGRGEPRFIAVG
YVDDTQFVRFDSDAASQRMEPRAPWIEQEGPEYWDGETRKVKAHSQTHRV
DLGTLRGYYNQSEAGSHTVQRMYGCDVGSDWRFLRGYHQYAYDGKDYIAL
KEDLRSWTAADMAAQTTKHKWEAAHVAEQLRAYLEGTCVEWLRRYLENGK
ETLQRTDAPKTHMTHHAVSDHEATLRCWALSFYPAEITLTWQRDGEDQTQ
DTELVETRPAGDGTFQKWAAVVVPSGQEQRYTCHVQHEGLPKPLTLRWEP
SSQPTIP [symbol] [symbol] [symbol] KFSRSADAPAYQQGQNQ
LYNELNLGRREEYDVLDKRRGRDPEMGGKPQRRKNPQEGLYNELQKDKMA
EAYSEIGMKGERRRGKGHDGLYQGLSTATKDTYDALHMQALPPR

Ectodomain=unformatted text

Bold/underlined=transmembrane

Italics=endodomain

An illustrative polypeptide for use in the present invention is shown as SEQ ID NO: 37

(HLA-A03-CAR)
SEQ ID NO: 37
MAVMAPRTLLLLLSGALALTQTWAGSHSMRYFFTSVSRPGRGEPRFIAVG
YVDDTQFVRFDSDAASQRMEPRAPWIEQEGPEYWDQETRNVKAQSQTDRV
DLGTLRGYYNQSEAGSHTIQIMYGCDVGSDGRFLRGYRQDAYDGKDYIAL
NEDLRSWTAADMAAQITKRKWEAAHEAEQLRAYLDGTCVEWLRRYLENGK
ETLQRTDPPKTHMTHHPISDHEATLRCWALGFYPAEITLTWQRDGEDQTQ
DTELVETRPAGDGTFQKWAAVVVPSGEEQRYTCHVQHEGLPKPLTLRWEL
SSQPTIP [symbol] [symbol] [symbol] KFSRSADAPAYQQGQNQL
YNELNLGRREEYDVLDKRRGRDPEMGGKPQRRKNPQEGLYNELQKDKMAE
AYSEIGMKGERRRGKGHDGLYQGLSTATKDTYDALHMQALPPR

Ectodomain=unformatted text

Bold/underlined=transmembrane

Italics=endodomain

An illustrative polypeptide for use in the present invention is shown as SEQ ID NO: 38

(HLA-B07-CAR)
SEQ ID NO: 38
MLVMAPRTVLLLLSAALALTETWAGSHSMRYFYTSVSRPGRGEPRFISVG
YVDDTQFVRFDSDAASPREEPRAPWIEQEGPEYWDRNTQIYKAQAQTDRE
SLRNLRGYYNQSEAGSHTLQSMYGCDVGPDGRLLRGHDQYAYDGKDYIAL
NEDLRSWTAADTAAQITQRKWEAAREAEQRRAYLEGECVEWLRRYLENGK
DKLERADPPKTHVTHHPISDHEATLRCWALGFYPAEITLTWQRDGEDQTQ
DTELVETRPAGDRTFQKWAAVVVPSGEEQRYTCHVQHEGLPKPLTLRWEP
SSQSTVP [symbol] [symbol] [symbol] KFSRSADAPAYQQ
GQNQLYNELNLGRREEYDVLDKRRGRDPEMGGKPQRRKNPQEGLYNELQ
KDKNIAEAYSEIGMKGERRRGKGHDGLYQGLSTATKDTYDALHMQALP
PR

Ectodomain=unformatted text

Bold/underlined=transmembrane

Italics=endodomain

An illustrative polypeptide for use in the present invention is shown as SEQ ID NO: 39

(HLA-B08-CAR)
SEQ ID NO: 39
MLVMAPRTVLLLLSAALALTETWAGSHSMRYFDTAMSRPGRGEPRFISVG
YVDDTQFVRFDSDAASPREEPRAPWIEQEGPEYWDRNTQIFKTNTQTDRE
SLRNLRGYYNQSEAGSHTLQSMYGCDVGPDGRLLRGHNQYAYDGKDYIAL
NEDLRSWTAADTAAQITQRKWEAARVAEQDRAYLEGTCVEWLRRYLENGK
DTLERADPPKTHVTHHPISDHEATLRCWALGFYPAEITLTWQRDGEDQTQ
DTELVETRPAGDRTFQKWAAVVVPSGEEQRYTCHVQHEGLPKPLTLRWEP
SSQSTVP [symbol] [symbol] KFSRSADAPAYQQGQNQL
YNELNLGRREEYDVLDKRRGRDPEMGGKPQRRKNPQEGLYNELQKDKMAE
AYSEIGMKGERRRGKGHDGLYQGLSTATKDTYDALHMQALPPR

Ectodomain=unformatted text

Bold/underlined=transmembrane

Italics=endodomain

An illustrative polypeptide for use in the present invention is shown as SEQ ID NO: 40

(HLA-B44-CAR)
SEQ ID NO: 40
MRVTAPRTLLLLLWGAVALTETWAGSHSMRYFYTAMSRPGRGEPRFITVG
YVDDTLFVRFDSDATSPRKEPRAPWIEQEGPEYWDRETQISKTNTQTYRE
NLRTALRYYNQSEAGSHIIQRMYGCDVGPDGRLLRGYDQDAYDGKDYIAL
NEDLSSWTAADTAAQITQRKWEAARVAEQDRAYLEGLCVESLRRYLENGK
ETLQRADPPKTHVTHHPISDHEVTLRCWALGFYPAEITLTWQRDGEDQTQ
DTELVETRPAGDRTFQKWAAVVVPSGEEQRYTCHVQHEGLPKPLTLRWEP
SSQSTVP [symbol] [symbol] [symbol] KFSRSADAPAYQQGONQL
YNELNLGRREEYDVLDKRRGRDPEMGGKPQRRKNPQEGLYNELQKDKMAE
AYSEIGMKGERRRGKGHDGLYQGLSTATKDTYDALHMQALPPR

Ectodomain=unformatted text

Bold/underlined=transmembrane

Italics=endodomain

An illustrative polypeptide for use in the present invention is shown as SEQ ID NO: 41

(HLA-001-CAR)
SEQ ID NO: 41
MRVMAPRTLILLLSGALALTETWACSHSMKYFFTSVSRPGRGEPRFISVG
YVDDTQFVRFDSDAASPRGEPRAPWVEQEGPEYWDRETQKYKRQAQTDRV
SLRNLRGYYNQSEAGSHTLQWMCGCDLGPDGRLLRGYDQYAYDGKDYIAL
NEDLRSWTAADTAAQITQRKWEAAREAEQRRAYLEGTCVEWLRRYLENGK
ETLQRAEHPKTHVTHHPVSDHEATLRCWALGFYPAEITLTWQWDGEDQTQ
DTELVETRPAGDGTFQKWAAVMVPSGEEQRYTCHVQHEGLPEPLTLRWEP
SSQPTIP [symbol] [symbol] KFSRSADAPAYQQGQNQLY
NELNLGRREEYDVLDKRRGRDPEMGGKPQRRKNPQEGLYNELQKDKMAEA
YSEIGMKGERRRGKGHDGLYQGLSTATKDTYDALHMQALPPR

A polypeptide sequence for use in the present invention may comprise the sequence shown as SEQ ID NO: 35-41 or a variant thereof having at least 80% sequence identity. The variant maintains ability to assemble and facilitate productive peptide presentation by the MHC class I complex at the surface of the cell and transmit an activating signal following binding of a TCR to the peptide/MHC complex comprising the polypeptide.

The variant of SEQ ID NO: 35-41 may have at least 80, 85, 90, 95, 98 or 99% sequence identity, provided that the variant maintains ability to assemble and facilitate productive peptide presentation by the MHC class I complex at the surface of the cell and transmit an activating signal following binding of a TCR to the peptide/MHC complex comprising the polypeptide.

In a further independent embodiment, the present invention provides an engineered polypeptide comprising an ectodomain from HLA-A, HLA-B or HLA-C that is linked to an intracellular signalling domain, such as a component of the CD3 complex as described herein.

The present invention further provides a polynucleotide encoding an engineered polypeptide comprising an ectodomain from HLA-A, HLA-B or HLA-C that is linked to an intracellular signalling domain, such as a component of the CD3 complex as described herein. The invention also provides a vector comprising said polynucleotide.

Further, the present invention provides a cell which comprises an engineered polypeptide comprising an ectodomain from HLA-A, HLA-B or HLA-C that is linked to an intracellular signalling domain, such as a component of the CD3 complex as described herein or a polynucleotide or a vector which encodes said engineered polypeptide.

HLA Class II

MHC class II molecules are restricted to professional antigen-presenting cells such as dendritic cells, mononuclear phagocytes, some endothelial cells, thymic epithelial cells and B cells. These cells are important in initiating immune responses by presenting class II peptides derived from extracellular proteins. Loading of MHC class II occurs by phagocytosis; extracellular proteins are endocytosed, digested in lysosomes and the epitopic peptide fragments are loaded onto MHC class II molecules before their migration to the cell surface.

In humans the MHC class II protein complex is encoded by the human leukocyte antigen gene complex (HLA). HLAs corresponding to MHC class II are HLA-DP, HLA-DM, HLA-DOA, HLA-DOB, HLA-DQ and HLA-DR.

However, expression of MHC class II molecules can be induced in nonprofessional APC (e.g. fibroblasts, epithelial cells, keratinocytes) in an environment rich in inflammatory cytokines, such as interferon γ.

Activated human T cells express MHC class II molecules of all isotypes (HLA-DR, HLA-DQ, and HLA-DP) on their surface. Expression of MHC class II molecules is found approximately 3 to 5 days after T-cell activation, which is a relative late event compared with the induction of a variety of other effector molecules after T-cell receptor (TCR)-triggering and co-stimulation. Since adoptively transferred immune effectors are expected to be activated at some point after infusion, expression of HLA class II can lead to allo-rejection.

HLA class II molecules are formed as two polypeptide chains: alpha and beta. These are typically highly polymorphic from one individual to another, although some haplotypes are much more common in certain populations than others.

Polypeptides for any haplotype or any combination of haplotypes may be used in the present invention including any of those recited in the table below:

TABLE 2
Exemplary common haplotypes.

HLA-DRB
HLA-DRB03
HLA-DRB15
HLA-DRB04
HLA-DRB07
HLA-DRB01

HLA-DR has very little polymorphism, making it particularly suitable for use in the present invention. In one embodiment, the at least one polypeptide capable of co-localizing the MHC class II polypeptide with the intracellular signalling domain comprises an ectodomain from HLA-DR and an intracellular signalling domain. Suitably, the at least one polypeptide may comprise an ectodomain from HLA-DRα and an intracellular signalling domain. Suitably, the at least one polypeptide may comprise an ectodomain from HLA-DRβ and an intracellular signalling domain.

An illustrative amino acid sequence of HLA class II histocompatibility antigen, DR α chain (which has UniProtKB accession number P01903) is shown as SEQ ID NO: 1:

SEQ ID NO: 1
MAISGVPVLGFFIIAVLMSA

[symbol]

[symbol]

[symbol]

[symbol]

[symbol]

[symbol]

[symbol]

[symbol]

[symbol]

[symbol]

[symbol]

[symbol]

[symbol]

[symbol]

NVVCA
LGLTVGLVGIIIGTIFIIKGVRKSNAAERRGPL

Bold underlined=ecotodomain of this HLADRα sequence corresponds to amino acid positions 26-216 of the sequence.

Suitably, the at least one polypeptide capable of co-localizing an MHC class II polypeptide with an intracellular signalling domain within the cell may comprise an ectodomain from HLA-DRα. Suitably, the at least one polypeptide capable of co-localizing an MHC class II polypeptide with an intracellular signalling domain within the cell may comprise an ectodomain from HLA-DRα as set forth SEQ ID NO: 1 (such as from about amino acid 26 to about amino acid 216 of SEQ ID NO: 1) or a variant thereof having at least 80% sequence identity.

An HLA-DRα sequence for use in the present invention may comprise the sequence shown as SEQ ID NO: 1 or a variant thereof having at least 80% sequence identity. The variant maintains ability to assemble with a β chain and facilitate productive peptide presentation by the MHC class II complex.

The variant of SEQ ID NO: 1 may have at least 80, 85, 90, 95, 98 or 99% sequence identity, provided that the variant maintains ability to assemble with a β chain and facilitate productive peptide presentation by the MHC class II complex.

An illustrative amino acid sequence of HLA class II histocompatibility antigen, DR β chain (which has UniProtKB accession number Q04826) is shown as SEQ ID NO: 2:

SEQ ID NO: 2
MRVTAPRILLLLLWGAVALT

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

VGIVAGLAVLAVVVIGAVVAAVMCRRKSSGGKGGSY
SQAACSDSAQGSDVSLTA

Bold underlined=the ecotodomain of this HLA-DRβ sequence and corresponds to amino acid positions 25-308 of the sequence.

Suitably, the at least one polypeptide capable of co-localizing an MHC class II polypeptide with an intracellular signalling domain within the cell may comprise an ectodomain from HLA-DRβ.

Suitably, the at least one polypeptide capable of co-localizing an MHC class II polypeptide with an intracellular signalling domain within the cell may comprise an ectodomain from HLA-DRβ as set forth in SEQ ID NO: 2 (such as from about amino acid 25 to about amino acid 308 of SEQ ID NO: 2) or a variant thereof having at least 80% sequence identity.

An HLA-DRβ sequence for use in the present invention may comprise the sequence shown as SEQ ID NO: 2 or a variant thereof having at least 80% sequence identity. The variant maintains ability to assemble with an α chain and facilitate productive peptide presentation by the MHC class II complex.

The variant of SEQ ID NO: 2 may have at least 80, 85, 90, 95, 98 or 99% sequence identity, provided that the variant maintains ability to assemble with an α chain and facilitate productive peptide presentation by the MHC class II complex.

HLA-DP and HLA-DQ have polymorphic α and β chains. Therefore one can select common HLA-DP or HLA-DQ a or β chain and restrict allogeneic production only from recipients with that haplotype. Suitably, the recipient may be homozygous for that haplotype. Wherein the recipient is not homozygous for the haplotype, two HLA-DP and two HLA-DQ (optionally in combination with HLA-DR e.g. HLA-DRα) may be used.

An illustrative amino acid sequence of HLA class II histocompatibility antigen, DP (which has UniProtKB accession number Q30058) is shown as SEQ ID NO: 3:

SEQ ID NO. 3
MVLQVSAAPRTVALTALLMVLLTSVVQG

[symbol]

[symbol]

[symbol]

[symbol]

[symbol]

[symbol]

[symbol]

[symbol]

[symbol]

[symbol]

[symbol]

[symbol]

[symbol]

TLTGAGGFVLGLIICGVGIFMHRRSKKVQRGSA

Italics=the transmembrane region and corresponds to amino acid positions 225 to 244

Bold underlined=the ecotodomain of this HLA-DP sequence and corresponds to amino acid positions 29-224 of the sequence

Suitably, the at least one polypeptide capable of co-localizing an MHC class II polypeptide with an intracellular signalling domain within the cell may comprise an ectodomain from HLA-DP. Suitably, the at least one polypeptide capable of co-localizing a MHC class II polypeptide with an intracellular signalling domain within the cell may comprise an ectodomain from HLA-DP as set forth SEQ ID NO: 3 (such as from about amino acid 29 to about amino acid 224 of SEQ ID NO: 3) or a variant thereof having at least 80% sequence identity.

An HLA-DP sequence for use in the present invention may comprise the sequence shown as SEQ ID NO: 3 or a variant thereof having at least 80% sequence identity. The variant maintains ability to assemble and facilitate productive peptide presentation by the MHC class II complex.

The variant of SEQ ID NO: 3 may have at least 80, 85, 90, 95, 98 or 99% sequence identity, provided that the variant maintains ability to assemble and facilitate productive peptide presentation by the MHC class II complex.

An illustrative amino acid sequence of HLA class II histocompatibility antigen, DQ (which has UniProtKB accession number 019764) is shown as SEQ ID NO: 4:

SEQ ID NO. 4

SWKKALRIPGDLRVATVTLMLAMLSSLLAEG

[symbol]

[symbol]

[symbol]

[symbol]

[symbol]

[symbol]

[symbol]

[symbol]

[symbol]

[symbol]

[symbol]

[symbol]

[symbol]

[symbol]

MLSGVGGFVLGLIFLGLGLII

Italics=the transmembrane region and corresponds to amino acid positions 229-249

Bold underlined=the ecotodomain of this HLA-DQ sequence and corresponds to amino acid positions 32-228 of the sequence.

Suitably, the at least one polypeptide capable of co-localizing an MHC class II polypeptide with an intracellular signalling domain within the cell may comprise an ectodomain from HLA-DQ. Suitably, the at least one polypeptide capable of co-localizing a MHC class II polypeptide with an intracellular signalling domain within the cell may comprise an ectodomain from HLA-DQ as set forth SEQ ID NO: 4 (such as from about amino acid 32 to about amino acid 228 of SEQ ID NO: 4) or a variant thereof having at least 80% sequence identity.

An HLA-DQ sequence for use in the present invention may comprise the sequence shown as SEQ ID NO: 4 or a variant thereof having at least 80% sequence identity. The variant maintains ability to assemble and facilitate productive peptide presentation by the MHC class II complex.

The variant of SEQ ID NO: 4 may have at least 80, 85, 90, 95, 98 or 99% sequence identity, provided that the variant maintains ability to assemble and facilitate productive peptide presentation by the MHC class II complex.

The sequences of MHC polypeptides are provided in the ImMunoGeneTics (IMGT) database (Lefranc, M.-P. et al., Nucleic Acids Res., 27:209-212 (1999); doi: 10.1093/nar/27.1.209).

The percentage identity between two polypeptide sequences may be readily determined by programs such as BLAST, which is freely available at http://blast.ncbi.nlm.nih.gov. Suitably, the percentage identity is determined across the entirety of the reference and/or the query sequence.

As used herein, “capable of co-localizing an MHC class I polypeptide or MHC class II polypeptide with an intracellular signalling domain within the cell” means that, when a reactive T-cell binds to a peptide/MHC complex on a cell of the present invention, the polypeptide co-localizes the MHC class I polypeptide or MHC class II polypeptide with the intracellular signalling domain such that the intracellular signalling domain transmits an activating signal in the cell of the present invention.

Suitably, the activating signal that is induced stimulates the cell of the present invention to deplete the reactive T cell which recognises the peptide/MHC complex.

Suitable methods for determining activation of the cytotoxic killing mechanisms in a cell include, but are not limited to, chromium release assays, flow-cytometry based killing assays, measuring cytokine release after effector and target encounter (e.g. by ELISA or cytokine bead array), demonstration of de-granulation or activation on effector cells after effector-target cell encounter by flow-cytometry. Depletion of allo-reactive T cells may be measured for example by flow cytometry.

In one embodiment, a cell according to the present invention comprises one or more (such as one, or two, or three) polypeptides selected from:

a polypeptide comprising an ectodomain from HLA-DR and an intracellular signalling domain;

a polypeptide comprising an ectodomain from HLA-DP and an intracellular signalling domain; and

a polypeptide comprising an ectodomain from HLA-DQ and an intracellular signalling domain.

Suitably, a cell may comprise:

a polypeptide comprising an ectodomain from HLA-DR and an intracellular signalling domain; and

a polypeptide comprising an ectodomain from HLA-DP and an intracellular signalling domain; and

a polypeptide comprising an ectodomain from HLA-DQ and an intracellular signalling domain.

Engineered MHC Class II Polypeptide

In some embodiments, the at least one polypeptide capable of co-localizing the MHC class II polypeptide with the intracellular signalling domain comprises an ectodomain from HLA-DR, HLA-DP or HLA-DQ and an intracellular signalling domain.

Such an engineered polypeptide comprises an MHC class II ectodomain and is capable of facilitating productive peptide presentation by the MHC class II complex on the cell surface. In addition, the polypeptide further comprises an intracellular signalling domain that is capable of transmitting an activating signal following binding of a TCR to the peptide/MHC complex which comprises the engineered polypeptide. The intracellular signalling domain may be an intracellular signalling domain as described herein.

A T-cell expressing such a polypeptide will selectively deplete any reactive T-cell which recognizes the selected peptide/MHC complex. In this way, selective immunosuppression against a known antigen can be executed by depletion of cognate T-cells. This advantageously enables a cell expressing the present MHC class II complexes to deplete endogenous, reactive T cells which recognise any peptide/MHC complex which is presented by the cell of the present invention.

In some embodiments the at least one polypeptide capable of co-localizing the MHC class II polypeptide with the intracellular signalling domain comprises an ectodomain from HLA-DR, HLA-DP or HLA-DQ, a transmembrane domain and an intracellular signalling domain. Suitably, the polypeptide may comprise a transmembrane domain located between the HLA ectodomain and the endodomain comprising an intracellular signalling domain.

The transmembrane domain may be any peptide domain that is capable of inserting into and spanning the cell membrane. A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (U.S. Pat. No. 7,052,906 B1 describes synthetic transmembrane components). For example, the transmembrane domain may comprise a hydrophobic alpha helix. The transmembrane domain may be derived from CD8alpha or CD28.

By way of example, the transmembrane domains of CD8-alpha and CD28 are shown as SEQ ID NO: 5 and SEQ ID NO: 6, respectively.

(CD8 alpha transmembrane domain)
SEQ ID NO: 5
IYIWAPLAGTCGVLLLSLVITLY
(CD28 alpha transmembrane domain)
SEQ ID NO: 6
FWVLVVVCGVLACYSLLVTVAFIIFWVR

CD79

CD79 is comprised of two chains, CD79α and CD79β which form a heterodimer on the surface of B cells. CD79α a/β assemble with membrane-bound immunoglobulin forming a complex with the B-cell receptor (BCR). CD79α and CD79β are members of the immunoglobulin superfamily and contain ITAM signalling motifs which enable B-cell signalling in response to cognate antigen recognition by the BCR.

CD79α and CD79β also associate with HLA class II, which allows HLA class II to signal through CD79 in an analogous way to membrane-bound immunoglobulin (Lang, P. et al. Science 291, 1537-1540 (2001) and Jin, L. et al. Immunol. Lett. 116,184-194 (2008).

In one aspect, the present invention provides a cell which comprises;

(i) a chimeric antigen receptor (CAR) or a transgenic T-cell receptor (TCR); and

(ii) at least one polypeptide capable of co-localizing an MHC class I polypeptide or an MHC class II polypeptide with an intracellular signalling domain within the cell; wherein the at least one polypeptide capable of co-localizing the MHC class I polypeptide or MHC class II polypeptide with the intracellular signalling domain is CD79 or a variant thereof.

The cell may comprise an engineered polypeptide which comprises CD79α or CD79β linked to an intracellular signalling domain. The cell may comprise two engineered polypeptides: one which comprises CD79α linked to an intracellular signalling domain; and one which comprises CD79β linked to an intracellular signalling domain.

An illustrative amino acid sequence of human CD79 α (which has UniProtKB accession number P11912) is shown as SEQ ID NO: 7:

SEQ ID NO. 7
MPGGPGVLQALPATIFLLFLLSAVYLGPGCQALWMHKVPASLMVSLGEDA
HFQCPHNSSNNANVTWWRVLHGNYTWPPEFLGPGEDPNGTLIIQNVNKSH
GGIYVCRVQEGNESYQQSCGTYLRVRQPPPRPFLDMGEGTKNRIITAEGI
ILLFCAVVPGTLLLFRKRWQNEKLGLDAGDEYEDENLYEGLNLDDCSMYE
DISRGLOGTYQDVGSLNIGDVQLEKP

Underlined=signal peptide (amino acids 1-32)

Bold=extracellular (amino acids 33-143)

No formatting=transmembrane domain (amino acids 144-165)

Italics=cytoplasmic domain (amino acids 166-226)

A CD79 α sequence for use in the present invention may comprise the sequence shown as SEQ ID NO: 7 or a variant thereof having at least 80% sequence identity. The variant maintains ability to assemble with HLA class I and/or HLA class II and facilitate signalling.

The variant of SEQ ID NO: 7 may have at least 80, 85, 90, 95, 98 or 99% sequence identity, provided that the variant maintains ability to assemble with HLA class I and/or HLA class II and facilitate signalling.

Suitably, the at least one polypeptide capable of co-localizing the MHC class I polypeptide or MHC class II polypeptide with the intracellular signalling domain may comprise an ectodomain of CD79a, a transmembrane domain and an intracellular signalling domain. Suitably, the at least one polypeptide may comprise an ectodomain of CD79α which corresponds to about amino acid 33 to about amino acid 143 of SEQ ID NO. 7.

Suitably, the at least one polypeptide may comprise a transmembrane domain of CD79α which corresponds to about amino acid 144 to about amino acid 165 of SEQ ID NO. 7.

Suitably, the at least one polypeptide may comprise an intracellular signalling domain of CD79α which corresponds to about amino acid 166 to about amino acid 226 of SEQ ID NO. 7.

Suitably, the at least one polypeptide capable of co-localizing the MHC class I polypeptide or MHC class II polypeptide with the intracellular signalling domain may be or comprise CD79a. Suitably, the at least one polypeptide capable of co-localizing the MHC class I polypeptide or MHC class II polypeptide with the intracellular signalling domain may be or comprise SEQ ID NO. 7.

An illustrative amino acid sequence of human CD79 β (which has UniProtKB accession number P40259) is shown as SEQ ID NO: 8:

SEQ ID NO. 8
MARLALSPVPSHWMVALLLLLSAEPVPAARSEDRYRNPKGSACSRIWQSP
RFIARKRGFTVKMHCYMNSASGNVSWLWKQEMDENPQQLKLEKGRMEESQ
NESLATLTIQGIRFEDNGIYFCQQKCNNTSEVYQGCGTELRVMGFSTLAQ
LKQRNTLKDGIIMIQTLLIILFIIVPIFLLLDKDDSKAGMEEDHTYEGLD
IDQTATYEDIVTLRTGEVKWSVGEHPGQE

Underlined=signal peptide (amino acids 1-28)

Bold=extracellular (amino acids 29-159)

No formatting=transmembrane domain (amino acids 160-180)

Italics=cytoplasmic (amino acids 181-229)

A CD79 β sequence for use in the present invention may comprise the sequence shown as SEQ ID NO: 8 or a variant thereof having at least 80% sequence identity. The variant maintains ability to assemble with HLA class I and/or HLA class II and facilitate signalling.

The variant of SEQ ID NO: 8 may have at least 80, 85, 90, 95, 98 or 99% sequence identity, provided that the variant maintains ability to assemble with HLA class I and/or HLA class II and facilitate signalling.

Suitably, the at least one polypeptide capable of co-localizing the MHC CLASS I polypeptide or MHC class II polypeptide with the intracellular signalling domain may comprise an ectodomain of CD796, a transmembrane domain and an intracellular signalling domain. Suitably, the at least one polypeptide may comprise an ectodomain of CD79 β which corresponds to about amino acid 29 to about amino acid 159 of SEQ ID NO. 8.

Suitably, the at least one polypeptide may comprise a transmembrane domain of CD79 β which corresponds to about amino acid 160 to about amino acid 180 of SEQ ID NO. 8.

Suitably, the at least one polypeptide may comprise an intracellular signalling domain of CD79 β which corresponds to about amino acid 181 to about amino acid 229 of SEQ ID NO. 8.

Suitably, the at least one polypeptide capable of co-localizing the MHC class I polypeptide or MHC class II polypeptide with the intracellular signalling domain may be or comprise CD79 β. Suitably, the at least one polypeptide capable of co-localizing the MHC class I polypeptide or MHC class II polypeptide with the intracellular signalling domain may be or comprise SEQ ID NO. 8.

Suitably, the at least one polypeptide capable of co-localizing the MHC class I polypeptide or MHC class II polypeptide with the intracellular signalling domain may be or comprise CD79α and CD79β. As will be apparent, the CD79α and CD79β may be provided as separate polypeptides. Suitably, the at least one polypeptide capable of co-localizing the MHC class I polypeptide or MHC class II polypeptide with the intracellular signalling domain may be or comprise SEQ ID NO. 7 or a variant thereof and SEQ ID NO. 8 or a variant thereof.

CD3 Linked Polypeptide

In other embodiments of the present invention, the at least one polypeptide capable of co-localizing the MHC class I polypeptide or MHC class II polypeptide with the intracellular signalling domain is an MHC class I polypeptide or an MHC class II polypeptide linked to a component of the TCR complex.

The cell may comprise:

(i) a chimeric antigen receptor (CAR) or a transgenic T-cell receptor (TCR); and

(ii) an engineered polypeptide which comprises an MHC class I polypeptide or MHC class II polypeptide linked to linked to a component of the CD3/TCR complex.

CD3 is a T-cell co-receptor that is involved in the activation of both cytotoxic T-cells and T-helper cells. It is formed of a protein complex composed of four distinct chains. As used herein, the term “CD3 complex” also includes the CD3 ζ-chain. In mammals, the complex contains a CD3γ chain, a CD3δ chain, and two CD3ε chains. These chains associate with the TCR to generate a TCR complex which is capable of producing an activation signal in T lymphocytes.

The CD3ζ, CD3γ, CD3δ, and CD3ε chains are highly related cell-surface proteins of the immunoglobulin superfamily containing a single extracellular immunoglobulin domain. The transmembrane region of the CD3 chains contain a number of aspartate residues are negatively charged, a characteristic that allows these chains to associate with the positively charged TCR chains. The intracellular tails of the CD3 molecules contain a single conserved motif known as an immunoreceptor tyrosine-based activation motif (ITAM), which is involved in TCR signalling.

The polypeptide linked to a component of the TCR complex is capable of assembling and facilitating productive peptide presentation by the MHC class I or MHC class II complex at the cell surface. In addition, the TCR/CD3 component is able to assemble with the TCR/CD3 complex. Hence, binding of a TCR to the peptide/MHC complex comprising the polypeptide linked to a component of the TCR complex will trigger signalling through the CD3/TCR complex.

The polypeptide may be linked to the TCR or a component of the CD3 complex. Suitably, the polypeptide may be linked to an engineered TCR polypeptide which lacks a variable domain. Suitably, the at least one polypeptide may be linked to a component of the CD3 complex. The component of the CD3 complex to which the at least one polypeptide is linked may be selected from CD3-zeta, CD3-epsilon, CD3-gamma and CD3-delta.

Suitably the component of the CD3 complex to which the at least one polypeptide is linked may be CD3-zeta.

Suitably the component of the CD3 complex to which the at least one polypeptide is linked may be CD3-epsilon.

Suitably the component of the CD3 complex to which the at least one polypeptide is linked may be CD3-gamma.

Suitably the component of the CD3 complex to which the at least one polypeptide is linked may be CD3-delta.

Examples of human CD3ζ, CD3γ, CD3δ and CD3ε amino acid sequences are shown as SEQ ID NO: 9-12, respectively. The CD3 polypeptide sequence for use in the present invention may comprise the sequence shown as one of SEQ ID NO: 9-12 or a variant thereof having at least 80% sequence identity. For example, the variant may have at least 80, 85, 90, 95, 98 or 99% sequence identity to one of SEQ ID NO: 9-12.

(CD3ζ-amino acids 1-21 provide a signal peptide
which maybe excluded,the transmembrane domain is
underlined)
SEQ ID NO: 9
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDICKAEAYSEIGMKGERRRGKGHDGLYQGLSTAT
KDTYDALHMQALPPR
(CD3γ-amino acids 1-22 provide a signal peptide
which maybe excluded
SEQ ID NO: 10)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN
(CD3δ-amino acids 1-21 provide a
signal peptide which may be excluded)
SEQ ID NO: 11
MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQVYQ
PLRDRDDAQYSHLGGNWARNK
(CD3ε-amino acids 1-22 provide a
signal peptide which maybe excluded)
SEQ ID NO: 12
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEP1RKGQRDLYS
GLNQRRI

The at least one polypeptide may be linked to the CD3 component by any suitable means. For example, the at least one polypeptide may be fused to the component of the CD3 complex by a linker peptide.

Suitable linker peptides are known in the art. For example, a range of suitable linker peptides are described by Chen et al. (Adv Drug Deliv Rev. 2013 October 15; 65(10): 1357-1369—see Table 3 in particular).

A suitable linker is an (SGGGG)n (SEQ ID NO: 13), which comprises one or more copies of SEQ ID NO: 13. For example, a suitable linker peptide is shown as SEQ ID NO: 14.

SEQ ID NO: 14-SGGGGSGGGGSGGGGS

Suitably, the at least one polypeptide may be linked to the ectodomain of the component of the CD3 complex. Suitably, the at least one polypeptide may be linked to the N-terminus of the component of the CD3 complex.

An illustrative polypeptide for use in the present
invention is shown as SEQ ID NO: 15.
METDILLLWVLLLWVPGSTGIKEEHVIIQAEFYLNPDQSGEFMFDFDGDE
IFHVDMAKKETVWRLEEFGRFASFEAQGALANIAVDKANLEIMTKRSNYT
PITNVPPEVTVLTNSPVELREPNVLICFIDKFTPPVVNVTWLRNGKPVTT
GVSETVFLPREDHLFRKFHYLPFLPSTEDVYDCRVEHWGLDEPLLKHWEF
DAPSPLPETTENVVCALGLTVGLVGIIIGTIFIIRVKFSRSADAPAYQQG
QNQLYNELNLGRREEYDVLDKRRGRDPEMGGKPRRKNPQEGLYNELQKDK
MAEAYSEIGMKGERRRGKGHDGLYQGLSIATKDTYDALHMQALPPRA

This polypeptide sequence comprises an ectodomain from HLA-DRα, a transmembrane domain an intracellular CD3-ζ endodomain.

An illustrative polypeptide for use in the present
invention is shown as SEQ ID NO: 16.
METDTLLLWVLLLWVPGSTGIKEEHVIIQAEFYLNPDQSGEFMFDFDGDE
IFHVDMAKKETVWRLEEFGRFASFEAQGALANIAVDKANLEIMTKRSNYT
PITNVPPEVTVLINSPVELREPNVLICFIDKFTPPVVNVTWLRNGKPVTT
GVSETVFLPREDHLFRKFHYLPFLPSTEDVYDCRVEHWGLDEPLLKHWEF
DAPSPLPETTENVVCALGLTVGLVGIIIGTIFIIKRGRKKLLYIFKQPFM
RPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPRA

This polypeptide sequence comprises an ectodomain from HLA-DRα, a transmembrane domain, a 41BB endodomain and an intracellular CD3-ζ endodomain.

An illustrative polypeptide for use
in the present invention is shown as
SEQ ID NO: 17.
METDTLLLWVLLLWVPGSTGIKEEHVIIQAEFYLN
PDQSGEFMFDFDGDEIFHVDMAKKETVWRLEEFGR
FASFEAQGALANIAVDKANLEIMTKRSNYTPITNV
PPEVTVLTNSPVELREPNVLICFIDKFTPPVVNVT
WLRNGKPVTTGVSETVFLPREDHLFRKFHYLPFLP
STEDVYDCRVEHWGLDEPLLKHWEFDAPSPLPETT
ENVVCALGLTVGLVGIIIGTIFIIRSKRSRLLHSD
YMNMTPRRPGPTRKHYQPYAPPRDFAAYRSRVKFS
RSADAPAYQQGQNQLYNELNLGRREEYDVLDKRRG
RDPEMGGKPRRKNPQEGLYNELQKDKMAEAYSEIG
MKGERRRGKGHDGLYQGLSTATKDTYDALHMQALP
PRA

This polypeptide sequence comprises an ectodomain from HLA-DRα, a transmembrane domain, a CD28 endodomain and an intracellular CD3-ζ endodomain.

An illustrative polypeptide for use
in the present invention is shown
as SEQ ID NO: 18.
METDILLLWVLLLWVPGSTGLWMHKVPASLMVSLG
EDAHFQCPHNSSNNANVTWWRVLHGNYTWPPEFLG
PGEDPNGTLIIQNVNKSHGGIYVCRVQEGNESYQQ
SCGTYLRVRQPPPRPFLDMGEGTKNRIITAEGIIL
LFCAVVPGTLLLFKRGRKKLLYIFKQPFMRPVQTT
QEEDGCSCRFPEEEEGGCELRKRWQNEKLGLDAGD
EYEDENLYEGLNLDDCSMYEDISRGLQGTYQDVGS
LNIGDVQLEKP

This polypeptide sequence comprises an ectodomain from CD79α, a 41BB domain and an endodomain from CD79.

An illustrative polypeptide for use
in the present invention is shown
as SEQ ID NO: 19.
METDTLLLWVLLLWVPGSTGARSEDRYRNPKGSAC
SRIWQSPRFIARKRGFTVKMHCYMNSASGNVSWLW
KQEMDENPQQLKLEKGRMEESQNESLATLTIQGIR
FEDNGIYFCQQKCNNTSEVYQCCGTELRVMGFSTL
AQLKQRNTLKDGIIMIQTLLIILFIIVPIFLLRSK
RSRLLHSDYMNMTPRRPGPTRKHYQPYAPPRDFAA
YRSLDKDDSKAGMEEDHTYEGLDIDQTATYEDIVT
LRTGEVKWSVGEHPGQE

This polypeptide sequence comprises an ectodomain from CD79β, a CD28 domain and an endodomain from CD79.

An illustrative polypeptide for use
in the present invention is shown
as SEQ ID NO: 20.
METDTLLLWVLLLWVPGSTGLWMHKVPASLMVSLG
EDAHFQCPHNSSNNANVTWWRVLHGNYTWPPEFLG
PGEDPNGTLIIQNVNKSHGGIYVCRVQEGNESYQQ
SCGTYLRVRQPPPRPFLDMGEGTKNRIITAEGIIL
LFCAVVPGTLLLFRSKRSRLLHSDYMNMTPRRPGP
TRKHYQPYAPPRDFAAYRSRKRWQNEKLGLDAGDE
YEDENLYEGLNLDDCSMYEDISRGLQGTYQDVGSL
NIGDVQLEKP

This polypeptide sequence comprises an ectodomain from CD79α, a CD28 domain and an endodomain from CD79.

An illustrative polypeptide for use
in the present invention is shown
as SEQ ID NO: 21.
METDTLLLWVLLLWVPGSTGARSEDRYRNPKGSAC
SRIWQSPRFIARKRGFTVKMHCYMNSASGNVSWLW
KQEMDENPQQLKLEKGRMEESQNESLATLTIQGIR
FEDNGIYFCQQKCNNTSEVYQGCGTELRVMGFSTL
AQLKQRNTLKDGIIMIQTLLIILFIIVPIFLLKRG
RKKLLYIFKQPFMRPVQTTQEEDGCSCRFPEEEEG
GCELLDKDDSKAGMEEDHTYEGLDIDQTATYEDIV
TLRTGEVKWSVGEHPGQE

This polypeptide sequence comprises an ectodomain from CD79β, a 41BB domain and an endodomain from CD79.

An illustrative polypeptide for use
in the present invention is shown
as SEQ ID NO: 22.
METDTLLLWVLLLWVPGSTGLWMHKVPASLMVSLG
EDAHFQCPHNSSNNANVTTNWRVLHGNYTWPPEFL
GPGEDPNGTLIIQNVNKSHGGIYVCRVQEGNESYQ
QSCGTYLRVRQPPPRPFLDMGEGTKNRIITAEGII
LLFCAVVPGTLLLFKRGRKKLLYIFKQPFMRPVQT
TQEEDGCSCRFPEEEEGGCELRVKFSRSADAPAYQ
QGQNQLYNELNLGRREEYDVLDKRRGRDPEMGGKP
RRKNPQEGLYNELQKDKMAEAYSEIGMKGERRRGK
GHDGLYQGLSTATKDTYDALHMQALPPRA

This polypeptide sequence comprises an ectodomain from CD79α, a 41BB domain and a CD3-zeta domain.

An illustrative polypeptide for use
in the present invention is shown
as SEQ ID NO: 23.
METDTLLLWVLLLWVPGSTGARSEDRYRNPKGSAC
SRIWQSPRFIARKRGFTVKMHCYMNSASGNVSWLW
KQEMDENPQQLKLEKGRMEESQNESLATLTIQGIR
FEDNGIYFCQQKCNNTSEVYQGCGTELRVMGFSTL
AQLKQRNTLKDIITAEGIILLFCAVVPGTLLLFKR
GRKKLLYIFKQPFMRPVQTTQEEDGCSCRFPEEEE
GGCELRVKFSRSADAPAYQQGQNQLYNELNLGRRE
EYDVLDKRRGRDPEMGGKPRRKNPQEGLYNELQKD
KMAEAYSEIGMKGERRRGKGHDGLYQGLSTATKDT
YDALHMQALPPRA

This polypeptide sequence comprises an ectodomain from CD79β, a 41BB domain and a CD3-zeta domain.

A polypeptide sequence for use in the present invention may comprise the sequence shown as SEQ ID NO: 15-23 or a variant thereof having at least 80% sequence identity. The variant maintains ability to assemble and facilitate productive peptide presentation by the MHC class II complex at the surface of the cell and transmit an activating signal following binding of a TCR to the peptide/MHC complex comprising the polypeptide.

The variant of SEQ ID NO: 15-23 may have at least 80, 85, 90, 95, 98 or 99% sequence identity, provided that the variant maintains ability to assemble and facilitate productive peptide presentation by the MHC class II complex at the surface of the cell and transmit an activating signal following binding of a TCR to the peptide/MHC complex comprising the polypeptide.

In a further independent embodiment, the present invention provides an engineered polypeptide comprising an ectodomain from HLA-DR, HLA-DP or HLA-DQ that is linked to an intracellular signalling domain, such as a component of the CD3 complex as described herein.

The present invention further provides a polynucleotide encoding an engineered polypeptide comprising an ectodomain from HLA-DR, HLA-DP or HLA-DQ that is linked to an intracellular signalling domain, such as a component of the CD3 complex as described herein. The invention also provides a vector comprising said polynucleotide.

Further, the present invention provides a cell which comprises an engineered polypeptide comprising an ectodomain from HLA-DR, HLA-DP or HLA-DQ that is linked to an intracellular signalling domain, such as a component of the CD3 complex as described herein or a polynucleotide or a vector which encodes said engineered polypeptide.

Intracellular Signalling Domain

The present invention involves providing at least one polypeptide capable of co-localizing an MHC class I polypeptide or MHC class II polypeptide with an intracellular signalling domain within the cell

An intracellular signalling domain as used herein refers to a signal-transmission portion of an endomain.

The intracellular signalling domain may be or comprise a T cell signalling domain.

The intracellular signalling domain may comprise one or more immunoreceptor tyrosine-based activation motifs (ITAMs). An ITAM is a conserved sequence of four amino acids that is repeated twice in the cytoplasmic tails of certain cell surface proteins of the immune system. The motif contains a tyrosine separated from a leucine or isoleucine by any two other amino acids, giving the signature YxxL/I. Two of these signatures are typically separated by between 6 and 8 amino acids in the tail of the molecule (YxxL/Ix₍₆₋₈₎YxxL/I).

ITAMs are important for signal transduction in immune cells. Hence, they are found in the tails of important cell signalling molecules such as the CD3 and ζ-chains of the T cell receptor complex, the CD79 alpha and beta chains of the B cell receptor complex, and certain Fc receptors. The tyrosine residues within these motifs become phosphorylated following interaction of the receptor molecules with their ligands and form docking sites for other proteins involved in the signalling pathways of the cell.

Preferably, the intracellular signalling domain component comprises, consists essentially of, or consists of the CD3-ζ endodomain, which contains three ITAMs. Classically, the CD3-ζ endodomain transmits an activation signal to the T cell after antigen is bound. However, in the context of the present invention, the CD3-ζ endodomain transmits an activation signal to the effector cell after its MHC complex interacts with a TCR on a neighbouring T cell.

The intracellular signalling domain may comprise additional co-stimulatory signalling. For example, 4-1BB (also known as CD137) can be used with CD3-ζ, or CD28 and OX40 can be used with CD3-ζ to transmit a proliferative/survival signal.

Accordingly, intracellular signalling domain may comprise the CD3-ζ endodomain alone, the CD3-ζ endodomain in combination with one or more co-stimulatory domains selected from 4-1BB, CD28 or OX40 endodomain, and/or a combination of some or all of 4-1BB, CD28 or OX40.

The endodomain may comprise one or more of the following: an ICOS endodomain, a CD2 endodomain, a CD27 endodomain, or a CD40 endodomain.

The endomain may comprise the sequence shown as SEQ ID NO: 24-27 or a variant thereof having at least 80% sequence identity. The variant having at least sequence identity maintains the signalling function of one of SEQ ID NO: 24-27.

The variant of one of the sequence shown as SEQ ID NO: 24-27 may have at least 80, 85, 90, 95, 98 or 99% sequence identity, provided that the variant sequence retains the capacity to transmit an activating signal to the cell.

The percentage identity between two polypeptide sequences may be readily determined by programs such as BLAST, which is freely available at http://blast.ncbi.nlm.nih.gov. Suitably, the percentage identity is determined across the entirety of the reference and/or the query sequence.

CD3-ζ endodomain
SEQ ID NO: 24
RVKFSRSADAPAYQQGQNQLYNELNLGRREEYDVL
DKRRGRDPEMGGKPRRKNPQEGLYNELQKDKMAEA
YSEIGMKGERRRGKGHDGLYQGLSTATKDTYDALH
MQALPPR
IBB and CD3-ζ endodomains
SEQ ID NO: 25
MGNSCYNIVATLLLVLNFERTRSLQDPCSNCPAGT
FCDNNRNQICSPCPPNSFSSAGGQRTCDICRQCKG
VFRTRKECSSTSNAECDCTPGFHCLGAGCSMCEQD
CKQGQELTKKGCKDCCFGTFNDQKRGICRPWTNCS
LDGKSVLVNGTKERDVVCGPSPADLSPGASSVTPP
APAREPGHSPQIISFFLALTSTALLFLLFFLTLRF
SVVKRGRKKLLYIFKQPFMRPVQTTQEEDGCSCRF
PEEEEGGCELRVKFSRSADAPAYQQGQNQLYNELN
LGRREEYDVLDKRRGRDPEMGGKPQRRKNPQEGLY
NELQKDKMAEAYSEIGMKGERRRGKGHDGLYQGLS
TATKDTYDALHMQALPPR
CD28 and CD3-ζ endodomains
SEQ ID NO: 26
SKRSRLLHSDYMNMTPRRPGPTRKHYQPYAPPRDF
AAYRSRVKFSRSADAPAYQQGQNQLYNELNLGRRE
EYDVLDKRRGRDPEMGGKPRRKNPQEGLYNELQKD
KMAEAYSEIGMKGERRRGKGHDGLYQGLSTATKDT
YDALHMQALPPR
CD28, OX40 and CD3-ζ endodomains
SEQ ID NO: 27
SKRSRLLHSDYMNMTPRRPGPTRKHYQPYAPPRDF
AAYRSRDQRLPPDAHKPPGGGSFRTPIQEEQADAH
STLAKIRVKFSRSADAPAYQQGQNQLYNELNLGRR
EEYDVLDKRRGRDPEMGGKPRRKNPQEGLYNELQK
DKMAEAYSEIGMKGERRRGKGHDGLYQGLSTATKD
TYDALHMQALPPR

Antigen-Binding Domain Linked to Signalling Domain

The engineered polypeptide of the present invention may comprise a binding domain which binds to an MHC class I polypeptide or an MHC class II polypeptide, linked to an intracellular signalling domain.

The binding domain may be or comprise and antibody or antibody-like molecule.

The term “antibody”, as used herein, refers to a polypeptide having an antigen binding site which comprises at least one complementarity determining region or CDR. The antibody may comprise 3 CDRs and have an antigen binding site which is equivalent to that of a single domain antibody (dAb), heavy chain antibody (VHH) or a nanobody. The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen.

A full-length antibody or immunoglobulin typically consists of four polypeptides: two identical copies of a heavy (H) chain polypeptide and two identical copies of a light (L) chain polypeptide. Each of the heavy chains contains one N terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. They are characterised by the same general structure constituted by relatively preserved regions called frameworks (FR) joined by three hyper-variable regions called complementarity determining regions (CDR). The term “complementarity determining region” or “CDR”, as used herein, refers to the region within an antibody that complements an antigen's shape. Thus, CDRs determine the protein's affinity and specificity for specific antigens. The CDRs of the two chains of each pair are aligned by the framework regions, acquiring the function of binding a specific epitope. Consequently, in the case of VH and VL domains both the heavy chain and the light chain are characterised by three CDRs, respectively CDRH1, CDRH2, CDRH3 and CDRL1, CDRL2, CDRL3.

The engineered polypeptide of the present invention may comprise a full-length antibody or an antigen-binding fragment thereof.

A full length antibody may, for example be an IgG, an IgM, an IgA, an IgD or an IgE.

An “antibody fragment” refers to one or more fragments or portions of an antibody that retain the ability to specifically bind to an antigen. The antibody fragment may comprise, for example, one or more CDRs, the variable region (or portions thereof), the constant region (or portions thereof), or combinations thereof. Examples of antibody fragments include, but are not limited to, a Fab fragment, a F(ab′)2 fragment, an Fv fragment, a single chain Fv (scFv), a domain antibody (dAb or VH), a single domain antibody (sdAb), a VHH, a nanobody, a diabody, a triabody, a trimerbody, and a monobody.

The engineered polypeptide of the invention may comprise an antigen-binding domain which is based on a non-immunoglobulin scaffold. These antibody-binding domains are also called antibody mimetics. Non-limiting examples of non-immunoglobulin antigen-binding domains include an affibody, a fibronectin artificial antibody scaffold, an anticalin, an affilin, a DARPin, a VNAR, an iBody, an affimer, a fynomeran, abdurin/nanoantibody, a centyrin, an alphabody, a nanofitin, and a D domain.

Several antibodies have been described which specifically bind MHC class I or MHC class II.

For example, WO05/023299, which is incorporated by reference, describes antibodies which bind MHC class II antigens, in particular antibodies against the HLA-DR alpha chain. Table 1 of that document contains the sequence characteristics of clones MS-GPC-1 (scFv-17), MS-GPC-6 (scFv-8A), MS-GPC-8 (scFv-B8) and MS-GPC-10 (scFv-E6) and FIG. 15 gives the VH and VL sequences for MS-GPC-1; MS-GPC-6; MS-GPC-8; MS-GPC-10; MS-GPC-8-6; MS-GPC-8-10; MS-GPC-8-17; MS-GPC-8-27; MS-GPC-8-6-13; MS-GPC-8-10-57; MS-GPC-8-27-41; MS-GPC-8-1; MS-GPC-8-9; MS-GPC-8-18; MS-GPC-8-6-2; MS-GPC-8-6-19; MS-GPC-8-6-27; MS-GPC-8-6-45; MS-GPC-8-6-47; MS-GPC-8-27-7; and MS-GPC-8-27-10.

The engineered polypeptide may comprise an MHC class II binding domain comprising one of these pairs of VH and VL sequences. In particular, engineered polypeptide may comprise an MHC class II binding domain based on the binder MS-GPC-8.

Andris et al (1995 Mol Immunol 32:14-15) describe six antibodies specific for human HLA class I and class II antigens including an antibody against HLA-DQ beta chain having the antibody clone name anti-HLAII/DQB1-MP1.

Watkins et al (2000 Tissue Antigens 55: 219-28) describe the isolation and characterisation of human monoclonal HLA-A2 antibodies. The antibody clones include: anti-HLA-A2/A28-3PF12, anti-HLA-A2/A28-3PC4 and anti-HLA-A2/A28-3PB2.

The engineered polypeptide of the present invention may comprise an MHC class I or MHC class II binding domain derived from any of these antibodies.

The engineered polypeptide may comprise a short flexible linker to introduce a chain-break. A chain break separate two distinct domains but allows orientation in different angles. Such sequences include the sequence SDP, and the sequence SGGGSDP (SEQ ID NO: 45).

The linker may comprise a serine-glycine linker, such as SGGGGS (SEQ ID NO: 46).

The engineered polypeptide may comprise a transmembrane domain, as defined above. For example, the engineered polypeptide may comprise the transmembrane domains of CD8-alpha or CD28 which are shown above as SEQ ID NO: 5 and SEQ ID NO: 6, respectively.

The engineered polypeptide comprises an intracellular signalling domain, as defined above. The engineered polypeptide may, for example, comprise the CD3 endodomain.

The engineered polypeptide may have the general structure:

MHC class I or II binding domain-transmembrane domain-intracellular signalling domain, or

MHC class I or II binding domain-linker-transmembrane domain-intracellular signalling domain.

In a further independent aspect, the present invention provides an engineered polypeptide which comprises a binding domain which is binds to an MHC class I polypeptide or an MHC class II polypeptide linked to an intracellular signalling domain as defined herein.

The present invention further provides a polynucleotide encoding a such an engineered polypeptide and a vector comprising said polynucleotide.

Further, the present invention provides a cell which comprises such an engineered polypeptide; or a polynucleotide or a vector which encodes such an engineered polypeptide.

CD4/CD8 Fusion Proteins

The engineered polypeptide of the present invention may comprise the MHC class II-binding domain of CD4 linked to an intracellular signalling domain, or MHC class I-binding domain of CD8 linked to an intracellular signalling domain.

CD4 and CD8 are co-receptors of the T cell receptor (TCR) and assists T cells in communicating with antigen-presenting cells.

CD4 (cluster of differentiation 4) is a glycoprotein found on the surface of immune cells such as T helper cells, monocytes, macrophages, and dendritic cells. CD4 is a member of the immunoglobulin superfamily, having four immunoglobulin domains (D1 to D4) that are exposed on the extracellular surface of the cell:
D1 which resembles an immunoglobulin variable (IgV) domain; and
D2, D3 and D4 which resemble immunoglobulin constant (IgC) domains.

The immunoglobulin variable (IgV) domain of D1 adopts an immunoglobulin-like β-sandwich fold with seven β-strands in 2 β-sheets. CD4 interacts with the β2-domain of MHC class II molecules through its D1 domain. T cells displaying CD4 molecules on their surface, therefore, are specific for antigens presented by MHC II, i.e. they are MHC class II-restricted.

The short cytoplasmic/intracellular tail (C) of CD4 contains a sequence of amino acids that allow it to recruit and interact with the tyrosine kinase Lck. When the extracellular D1 domain of CD4 binds to the β2 region of MHC class II, the resulting close proximity between the TCR complex and CD4 allows the tyrosine kinase Lck bound to the cytoplasmic tail of CD4 to phosphorylate tyrosine residues of immunoreceptor tyrosine activation motifs (ITAMs) on the cytoplasmic domains of CD3 to amplify the signal generated by the TCR. Phosphorylated ITAMs on CD3 recruit and activate SH2 domain-containing protein tyrosine kinases (PTK), such as ZAP70, to further mediate downstream signalling through tyrosine phosphorylation. These signals lead to the activation of transcription factors, including NF-κB, NFAT, AP-1, to promote T cell activation.

The amino acid sequence for human CD4 is available from UniProt, Accession No. P01730. The engineered polypeptide of the present invention may comprise the D1 domain of CD4, which has the sequence shown as SEQ ID No. 47. The positions of Gln40 and Thr45 are shown in bold and underlined.

(CD4 D1 domain)
SEQ ID No. 47
KKVVLGKKGDTVELTCTASQKKSIQFHWKNSN [symbol] IK
ILGNQGSFL [symbol] KGPSKLNDRADSRRSLWDQGNFPLI
IKNLKIEDSDTYICEVEDQKEEVQLLVFGL

The engineered polypeptide may comprise a variant D1 domain of CD4 comprising one or more amino acid mutations which increase the its binding affinity for the β2 region of MHC class II compared to the wild-type D1 domain.

For example, Wang et al (2011, PNAS 108:15960-15965) describe the affinity maturation of human CD4 by yeast surface display to increase the affinity of CD4 for HLA-DR1. It was found that a CD4 variant bearing the substitution mutations Gln40Tyr and Thr45Trp bound to HLA-DR1 with KD=8.8 μM compares with >400 μM for wild-type CD4.

The engineered polypeptide may comprise a variant D1 domain of CD4 comprising amino acid mutation(s) at position Gln40 and/or Thr45 with reference to the sequence shown as SEQ ID No. 47.

The engineered polypeptide may comprise a variant D1 domain of CD4 comprising amino acid substitution(s) Gln40Tyr and/or Thr45Trp with reference to the sequence shown as SEQ ID No. 47.

CD8 (cluster of differentiation 8) co-receptor is predominantly expressed on the surface of cytotoxic T cells, but can also be found on natural killer cells, cortical thymocytes, and dendritic cells. There are two isoforms CD8, alpha and beta, each encoded by a different gene.

To function, CD8 forms a dimer, consisting of a pair of CD8 chains. The most common form of CD8 is composed of a CD8-α and CD8-β chain, but homodimers of the CD8-α chain are also expressed on some cells. CD8-α and CD8-β are both members of the immunoglobulin superfamily having an immunoglobulin variable (IgV)-like extracellular domain connected to the membrane by a thin stalk, and an intracellular tail.

The extracellular IgV-like domain of CD8-α interacts with the α3 portion of the Class I MHC molecule. The main recognition site is a flexible loop at the α3 domain of an MHC molecule located between residues 223 and 229. Binding of CD8-α to MHC class I keeps the T cell receptor of the cytotoxic T cell and the target cell bound closely together during antigen-specific activation. The cytoplasmic tails of the CD8 co-receptor interact with Lck (lymphocyte-specific protein tyrosine kinase). Once the T cell receptor binds its specific antigen, Lck phosphorylates the cytoplasmic CD3 and ζ-chains of the TCR complex which initiates a cascade of phosphorylation eventually leading to activation of transcription factors like N FAT, NF-κB, and AP-1.

The engineered polypeptide of the present invention may comprise the IgV-like domain from CD8-α.

The amino acid sequence for human CD8a is available from UniProt, Accession No. P01732. The engineered polypeptide of the present invention may comprise the Ig-like V-type domain of CD8, which comprises amino acid residues 22-135 of this sequence and has the sequence shown as SEQ ID No. 48.

(CD8α Ig-like V-type domain)
SEQ ID No. 48
SQFRVSPLDRTWNLGETVELKCQVLLSNPTSGCSW
LFQPRGAAASPTFLLYLSQNKPKAAEGLDTQRFSG
KRLGDTFVLTLSDFRRENEGYYFCSALSNSIMYFS
HFVPVFLPA

The engineered polypeptide may comprise a variant CD8a Ig-like V-type domain comprising one or more amino acid mutations which increase the its binding affinity for the α3 portion of a Class I MHC molecule compared to the wild-type CD8α domain.

For example, high affinity mutants of CD8α may be generated and characterised using the in vitor evolution method described by Wang et al (2011, PNAS 108:15960-15965).

The engineered polypeptide may comprise a dimeric form of CD8. Devine et al (1999, J. Immunol. 162:846-851) describe a molecule which comprises two CD8α Ig domains linked via the carboxyl terminal of one to the amino terminal of the other by means of a peptide spacer. A peptide spacer of 20 amino acids of 4 repeating units of GGGGS (SEQ ID No. 59) was used to allow the 2 IG-like domains to adopt the correct confirmation.

The engineered polypeptide may comprise a CD8αα homodimer, as described in Devine et al 1999. The CD8αα homodimer may have the sequence shown as SEQ ID No. 60.

(CD8αα homodimer)
SEQ ID No. 60
SQFRVSPLDRTWNLGETVELKCQVLLSNPTSGCSW
LFQPRGAAASPTFLLYLSQNKPKAAEGLDTQRFSG
KRLGDTFVLTLSDFRRENEGYYFCSALSNSIMYFS
HFVPVFLPAGGGGSGGGGSGGGGSGGGGSSQFRVS
PLDRTWNLGETVELKCQVLLSNPTSGCSWLFQPRG
AAASPTFLLYLSQNKPKAAEGLDTQRFSGKRLGDT
FVLTLSDFRRENEGYYFCSALSNSIMYFSHFVPVF
LPA

The engineered polypeptide may comprise a CD8αβ heterodimer. For example, the engineered polypeptide may comprise an CD8α Ig-like V-type domain having the sequence shown as SEQ ID No. 48 joined to a an CD8β Ig-like V-type domain by a peptide spacer. The peptide spacer may be from 10 to 20, for example between 15 and 25 amino acids in length. The peptide spacer may be approximately 20 amino acids in length. The peptide spacer may comprise 4 repeating units of GGGGS (SEQ ID No. 59), as for the CD8αα homodimer described by Devine et al 1999.

The amino acid sequence for the CD8β Ig-like V-type domain is shown below as SEQ ID No. 61.

(CD8β Ig-like V-type domain)
SEQ ID No. 61
LQQTPAYIKVQTNKMVMLSCEAKISLSNMRIYWLR
QRQAPSSDSHHEFLALWDSAKGTIHGEEVEQEKIA
VFRDASRFILNLTSVKPEDSGIYFCMIVGSPELTF
GKGTQL

The engineered polypeptide may comprise a CD8αβ heterodimer in which the CD8α and CD8β domains are in either order in the construct, i.e. CD8αβ or CD8βα.

The engineered polypeptide may comprise a short flexible linker between the CD8α monomer, the CD8αα homodimer or the CD8αβ heterodimer and the stalk and/or transmembrane domain to introduce a chain-break. A chain break separate two distinct domains but allows orientation in different angles. Such sequences include the sequence SDP, and the sequence SGGGSDP (SEQ ID NO: 45).

The linker may comprise a serine-glycine linker, such as SGGGGS (SEQ ID NO: 46).

The engineered polypeptide may comprise a transmembrane domain, as defined above. For example, the engineered polypeptide may comprise the transmembrane domains of CD8-alpha or CD28 which are shown above as SEQ ID NO: 5 and SEQ ID NO: 6, respectively.

The engineered polypeptide comprises an intracellular signalling domain, as defined above. The engineered polypeptide may, for example, comprise the CD3ζ endodomain.

The engineered polypeptide may have the general structure:

CD4 D1 domain-linker-transmembrane domain-intracellular signalling domain;

CD8α Ig-like V-type domain-linker-transmembrane domain-intracellular signalling domain;

CD8αα homodimer-linker-transmembrane domain-intracellular signalling domain; or

CD8αβ homodimer-linker-transmembrane domain-intracellular signalling domain

In a further independent aspect, the present invention provides an engineered polypeptide which comprises the MHC class II-binding domain of CD4 linked to an intracellular signalling domain, or MHC class I-binding domain of CD8 linked to an intracellular signalling domain as defined herein.

The present invention further provides a polynucleotide encoding a such an engineered polypeptide and a vector comprising said polynucleotide.

Further, the present invention provides a cell which comprises such an engineered polypeptide; or a polynucleotide or a vector which encodes such an engineered polypeptide.

Bi-Specific Polypeptides

In a further embodiment of the present invention, the polypeptide capable of co-localizing the MHC class I polypeptide or an MHC class II polypeptide with an intracellular signalling domain may be a bispecific polypeptide which comprises:

(a) a first binding domain which is binds to an MHC class I polypeptide or an MHC class II polypeptide

(b) a second binding domain which is capable of binding to a polypeptide comprising an intracellular signalling domain or a component of the CD3 complex.

The bispecific polypeptide may be membrane-tethered.

When expressed by the cell or on the cell surface, the present bispecific molecule co-localises MHC class I or II and the TCR, and facilitates TCR signalling in a cell of the invention following binding of a TCR on a different T cell to the peptide/MHC complex bound by the bispecific molecule.

Bispecific molecules have been developed in a number of different formats. One of the most common is a fusion consisting of two single-chain variable fragments (scFvs) of different antibodies.

The first and/or second binding domains of the bispecific molecule may be antibody or immunoglobulin based binding domains.

As used herein, “antibody” means a polypeptide having an antigen binding site which comprises at least one complementarity determining region CDR. The antibody may comprise 3 CDRs and have an antigen binding site which is equivalent to that of a domain antibody (dAb). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen. The antibody may be a whole immunoglobulin molecule or a part thereof such as a Fab, F(ab)′2, Fv, single chain Fv (ScFv) fragment, Nanobody or single chain variable domain (which may be a VH or VL chain, having 3 CDRs). The antibody may be a bifunctional antibody. The antibody may be non-human, chimeric, humanised or fully human.

Alternatively, the first and/or second binding domains of the present bispecific molecule may comprise domains which are not derived from or based on an immunoglobulin. A number of “antibody mimetic” designed repeat proteins (DRPs) have been developed to exploit the binding abilities of non-antibody polypeptides. Such molecules include ankyrin or leucine-rich repeat proteins e.g. DARPins (Designed Ankyrin Repeat Proteins), Anticalins, Avimers and Versabodies.

The first binding domain of the present bispecific molecule is capable of binding to a MCH class I or MHC class II polypeptide.

As mentioned above several antibodies have been described which specifically bind MHC class I or MHC class II.

For example, WO05/023299, which is incorporated by reference, describes antibodies which bind MHC class II antigens, in particular antibodies against the HLA-DR alpha chain. Table 1 of that document contains the sequence characteristics of clones MS-GPC-1 (scFv-17), MS-GPC-6 (scFv-8A), MS-GPC-8 (scFv-B8) and MS-GPC-10 (scFv-E6) and FIG. 15 gives the VH and VL sequences for MS-GPC-1; MS-GPC-6; MS-GPC-8; MS-GPC-10; MS-GPC-8-6; MS-GPC-8-10; MS-GPC-8-17; MS-GPC-8-27; MS-GPC-8-6-13; MS-GPC-8-10-57; MS-GPC-8-27-41; MS-GPC-8-1; MS-GPC-8-9; MS-GPC-8-18; MS-GPC-8-6-2; MS-GPC-8-6-19; MS-GPC-8-6-27; MS-GPC-8-6-45; MS-GPC-8-6-47; MS-GPC-8-27-7; and MS-GPC-8-27-10.

The bispecific polypeptide may comprise an MHC class II binding domain comprising one of these pairs of VH and VL sequences. In particular, bispecific polypeptide may comprise an MHC class II binding domain based on the binder MS-GPC-8.

Andris et al (1995 Mol Immunol 32:14-15) describe six antibodies specific for human HLA class I and class II antigens including an antibody against HLA-DQ beta chain having the antibody clone name anti-HLAII/DQB1-MP1.

Watkins et al (2000 Tissue Antigens 55: 219-28) describe the isolation and characterisation of human monoclonal HLA-A2 antibodies. The antibody clones include: anti-HLA-A2/A28-3PF12, anti-HLA-A2/A28-3PC4 and anti-HLA-A2/A28-3PB2.

The bispecific polypeptide of the present invention may comprise an MHC class I or MHC class II binding domain derived from any of these antibodies.

The second domain of the present bispecific molecule is capable of binding to a polypeptide comprising an intracellular signalling domain or a component of the CD3 complex. In particular, the second domain may be capable of binding CD3 on the T-cell surface. In this respect, the second domain may comprise a CD3 or TCR-specific antibody or part thereof.

The second domain may comprise the complementarity determining regions (CDRs) from the scFv sequence shown as SEQ ID NO: 49.

The second domain may comprise a scFv sequence, such as the one shown as SEQ ID NO: 49. The second domain may comprise a variant of such a sequence which has at least 80% sequence identity and binds CD3.

The second domain may comprise an antibody or part thereof which specifically binds CD3, such as OKT3, WT32, anti-leu-4, UCHT-1, SPV-3TA, TR66, SPV-T3B or affinity tuned variants thereof.

The second domain of the bispecific molecule of the invention may comprise all or part of the monoclonal antibody OKT3, which was the first monoclonal antibody approved by the FDA. OKT3 is available from ATCC CRL 8001. The antibody sequences are published in U.S. Pat. No. 7,381,803.

The second domain may comprise one or more CDRs from OKT3. The second binding domain may comprise CDR3 from the heavy-chain of OKT3 and/or CDR3 from the light chain of OKT3. The second binding domain may comprise all 6 CDRs from OKT3, as shown below.

Heavy Chain
CDR1:
(SEQ ID NO: 50)
KASGYTFTRYTMH
CDR2:
(SEQ ID NO: 51)
INPSRGYTNYNQKFKD
CDR3:
(SEQ ID NO: 52)
YYDDHYCLDY
Light Chain
CDR1:
(SEQ ID NO: 53)
SASSSVSYMN
CDR2:
(SEQ ID NO: 54)
RWIYDTSKLAS
CDR3:
(SEQ ID NO: 55)
QQWSSNPFT

The second binding domain may comprise a scFv which comprises the CDR sequences from OKT3. The second binding domain may comprise the scFv sequence shown below as SEQ ID NO: 49 or 56 or a variant thereof having at least 80% sequence identity, which retains the capacity to bind CD3.

SEQ ID NO: 49
QVQLQQSGAELARPGASVKMSCKASGYTFTRYTMH
WVKQRPGQGLEWIGYINPSRGYTNYNQKFKDKATL
ITDKSSSTAYMQLSSLISEDSAVYYCARYYDDHYC
LDYWGQGTTLTVSSSGGGGSGGGGSGGGGSQIVLT
QSPAIMSASPGEKVIMICSASSSVSYMNWYQQKSG
TSPKRWIYDTSKLASGVPAHFRGSGSGTSYSLTIS
GMEAEDAATYYCQQWSSNPFTFGSGTKLEINR
SEQ ID NO: 56
QIVLTQSPAIMSASPGEKVTMTCSASSSVSYMNWY
QQKSGTSPKRWIYDTSKLASGVPAHFRGSGSGTSY
SLTISGMEAEDAATYYCQQWSSNPFTFGSGTKLEI
NRSSSGGGGSGGGGSGGGGSQVQLQQSGAELARPG
ASVKMSCKASGYTFTRYTMHWVKQRPGQGLEWIGY
INPSRGYTNYNQKFKDKATLTTDKSSSTAYMQLSS
LTSEDSAVYYCARYYDDHYCLDYWGQGTTLTVSS

SEQ ID NO: 49 and 56 provide alternative architectures of an scFV suitable for use in the present invention. SEQ ID NO: 49 is provided as a VL-VH arrangement. SEQ ID NO: 56 is provided as a VH-VL arrangement.

A variant sequence from SEQ ID NO: 49 or 56 may have at least 80, 85, 90, 95, 98 or 99% sequence identity and have equivalent or improved CD3 binding capabilities as the sequence shown as SEQ ID NO: 49 or 56.

The bispecific molecule of the present invention may comprise a spacer sequence to connect the first domain with the second domain and spatially separate the two domains.

For example, the first and second binding domains may be connected via a short five residue peptide linker (GGGGS).

The spacer sequence may, for example, comprise an IgG1 hinge or a CD8 stalk. The linker may alternatively comprise an alternative linker sequence which has similar length and/or domain spacing properties as an IgG1 hinge or a CD8 stalk.

The spacer may be a short spacer, for example a spacer which comprises less than 100, less than 80, less than 60 or less than 45 amino acids. The spacer may be or comprise an IgG1 hinge or a CD8 stalk or a modified version thereof.

Examples of amino acid sequences for these linkers are given below:

(IgG1 hinge):
SEQ ID NO: 57
AEPKSPDKTHTCPPCPKDPKSGGGGS
(CD8 stalk):
SEQ ID NO: 58
TTIPAPRPPTPAPTIASQPLSLRP
EACRPAAGGAVHIRGLDFACD

The CD8 stalk has a sequence such that it may induce the formation of homodimers. If this is not desired, one or more cysteine residues may be substituted or removed from the CD8 stalk sequence. The bispecific molecule of the invention may include a spacer which comprises or consists of the sequence shown as SEQ ID NO: 58 or a variant thereof having at least 80, 85, 90, 95, 98 or 99% sequence identity, provided that the variant sequence is a molecule which causes approximately equivalent spacing of the first and second domains and/or that the variant sequence causes homodimerization of the bispecific molecule.

The bispecific molecule of the invention may have the general formula:
First domain-spacer-second domain.

The spacer may also comprise one or more linker motifs to introduce a chain-break. A chain break separate two distinct domains but allows orientation in different angles. Such sequences include the sequence SDP, and the sequence SGGGSDP (SEQ ID NO: 45).

The linker may comprise a serine-glycine linker, such as SGGGGS (SEQ ID NO: 46).

The spacer may cause the bispecific molecule to form a homodimer, for example due to the presence of one or more cysteine residues in the spacer, which can for a di-sulphide bond with another molecule comprising the same spacer.

The bispecific molecule may be membrane-tethered. In other words, the bispecific molecule may comprise a transmembrane domain such that it is localised to the cell membrane following expression in the cell of the present invention.

By way of example, the transmembrane domain may a transmembrane domain as described herein. For example, the transmembrane domain may comprise a hydrophobic alpha helix. The transmembrane domain may be derived from CD8alpha or CD28.

By way of example, the transmembrane domains of CD8alpha and CD28 are shown herein as SEQ ID NO: 5 and SEQ ID NO: 6, respectively.

The bispecific molecule of the invention may have the general formula:

First domain-spacer-second domain-transmembrane domain; or

Transmembrane domain-first domain-spacer-second domain.

In a further independent aspect, the present invention provides a bispecific molecule comprising:

(a) a first binding domain which is binds to an MHC class I polypeptide or an MHC class II polypeptide

(b) a second binding domain which is capable of binding to a polypeptide comprising an intracellular signalling domain or a component of the CD3 complex as described herein.

The present invention further provides a polynucleotide encoding a bispecific molecule comprising: (a) a first binding domain which is binds to an MHC class I polypeptide or an MHC class II polypeptide

(b) a second binding domain which is capable of binding to a polypeptide comprising an intracellular signalling domain or a component of the CD3 complex, as described herein. The invention also provides a vector comprising said polynucleotide.

Further, the present invention provides a cell which comprises a bispecific molecule as described herein; or a polynucleotide or a vector which encodes said bispecific molecule.

Signal Peptide

The present polypeptide capable of co-localizing an MHC class I polypeptide or an MHC class II polypeptide with an intracellular signalling domain within the cell may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

Cell

The cell of the present invention may be an immune effector cell, such as a T-cell, a natural killer (NK) cell or a cytokine induced killer cell.

The T cell may be an alpha-beta T cell. The T cell may be a gamma-delta T cell.

The cell may be derived from a patient's own peripheral blood (1st party), or in the setting of a haematopoietic stem cell transplant from donor peripheral blood (2nd party), or peripheral blood from an unconnected donor (3rd party). T or NK cells, for example, may be activated and/or expanded prior to being transduced with nucleic acid molecule(s) encoding the polypeptides of the invention, for example by treatment with an anti-CD3 monoclonal antibody.

Alternatively, the cell may be derived from ex vivo differentiation of inducible progenitor cells or embryonic progenitor cells to T cells. Alternatively, an immortalized T-cell line which retains its lytic function may be used.

The cell may be a haematopoietic stem cell (HSC). HSCs can be obtained for transplant from the bone marrow of a suitably matched donor, by leukopheresis of peripheral blood after mobilization by administration of pharmacological doses of cytokines such as G-CSF [peripheral blood stem cells (PBSCs)], or from the umbilical cord blood (UCB) collected from the placenta after delivery. The marrow, PBSCs, or UCB may be transplanted without processing, or the HSCs may be enriched by immune selection with a monoclonal antibody to the CD34 surface antigen

Chimeric Antigen Receptor

Classical CARs, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, as shown in the following Table. The antigen-binding domain used in the present invention may be a domain which is capable of binding a TAA as indicated therein.

TABLE 3

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The antigen-binding domain may comprise a proliferation-inducing ligand (APRIL) which binds to B-cell membrane antigen (BCMA) and transmembrane activator and calcium modulator and cyclophilin ligand interactor (TACI). A CAR comprising an APRIL-based antigen-binding domain is described in WO2015/052538.

Transmembrane Domain

The transmembrane domain is the sequence of a classical CAR that spans the membrane. It may comprise a hydrophobic alpha helix. The transmembrane domain may be derived from CD28, which gives good receptor stability.

Spacer Domain

The CAR may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Intracellular Signalling Domain

The intracellular signalling domain is the signal-transmission portion of a classical CAR.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together.

Transgenic T-Cell Receptor

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using vectors. For example the genes for engineered TCRs may be reintroduced into autologous T cells and transferred back into patients for T cell adoptive therapies. Such teterologous' TCRs may also be referred to herein as ‘transgenic TCRs’.

Nucleic Acid Construct/Kit of Nucleic Acid Sequences

The present invention provides a nucleic acid sequence which comprises: (i) a first nucleic acid sequence which encodes a chimeric antigen receptor (CAR) or a transgenic TCR; and (ii) at least one polypeptide capable of co-localizing an MHC class I polypeptide or MHC class II polypeptide with an intracellular signalling domain within the cell as defined herein.

The nucleic acid construct may comprise:

(i) a first nucleic acid sequence which encodes a chimeric antigen receptor (CAR) or a transgenic TCR; and

(ii) a second nucleic acid sequence which encodes an engineered polypeptide as defined herein or a bispecific polypeptide as defined herein.

The present invention further provides a kit comprising nucleic acid sequences according to the present invention. For example, the kit may comprise (i) a first nucleic acid sequence which encodes a chimeric antigen receptor (CAR) or a transgenic TCR; and (ii) a second nucleic acid sequence which encodes at least one polypeptide capable of co-localizing an MHC class I polypeptide or MHC class II polypeptide with an intracellular signalling domain within the cell as defined herein.

The kit of nucleic acid sequences may comprise:

(i) a first nucleic acid sequence which encodes a chimeric antigen receptor (CAR) or a transgenic TCR; and

(ii) a second nucleic acid sequence which encodes an engineered polypeptide as defined herein or a bispecific polypeptide as defined herein.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Co-Expression Site

A co-expression site is used herein to refer to a nucleic acid sequence enabling co-expression of both (i) a CAR or a TCR; and (ii) at least one polypeptide capable of co-localizing an MHC class I polypeptide or an MHC class II polypeptide with an intracellular signalling domain within the cell within the cell.

The co-expression site may be a nucleic acid sequence enabling co-expression of both (i) a first nucleic acid sequence which encodes a chimeric antigen receptor (CAR) or a transgenic TCR; and

(ii) a second nucleic acid sequence which encodes an engineered polypeptide or a bispecific polypeptide as defined herein.

The co-expression site may be a sequence encoding a cleavage site, such that the nucleic acid construct produces comprises the two polypeptides joined by a cleavage site(s). The cleavage site may be self-cleaving, such that when the polypeptide is produced, it is immediately cleaved into individual peptides without the need for any external cleavage activity.

The cleavage site may be any sequence which enables two (or more) polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al., 2001) as above.

The co-expressing sequence may be an internal ribosome entry sequence (IRES). The co-expressing sequence may be an internal promoter.

Vector

The present invention also provides a vector, or kit of vectors which comprises one or more nucleic acid sequence(s) or nucleic acid construct(s) of the invention. Such a vector may be used to introduce the nucleic acid sequence(s) or construct(s) into a host cell so that it expresses (i) a chimeric antigen receptor (CAR) or a transgenic TCR; and (ii) an engineered polypeptide or a bispecific polypeptide as defined herein.

The vector(s) may, for example, be a plasmid or a viral vector, such as a retroviral vector or a lentiviral vector, or a transposon based vector or synthetic mRNA.

The vector may be capable of transfecting or transducing a cell.

Pharmaceutical Composition

The present invention also relates to a pharmaceutical composition containing a plurality of cells, a nucleic acid construct, a first nucleic acid sequence and a second nucleic acid sequence; a vector or a first and a second vector of the present invention. In particular, the invention relates to a pharmaceutical composition containing a plurality of cells according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:

(i) isolating a cell-containing sample;

(ii) transducing or transfecting such cells with a nucleic acid construct, kit of nucleic acid sequences, vector or kit of vectors provided by the present invention;

(iii) administering the cells from (ii) to a subject.

The present invention provides a cell, a nucleic acid construct, a first nucleic acid sequence and a second nucleic acid sequence, a vector, or a first and a second vector of the present invention for use in treating and/or preventing a disease. In particular the present invention provides a cell of the present invention for use in treating and/or preventing a disease

The invention also relates to the use of a cell, a nucleic acid construct, a first nucleic acid sequence and a second nucleic acid sequence, a vector, or a first and a second vector of the present invention of the present invention in the manufacture of a medicament for the treatment and/or prevention of a disease. In particular, the invention relates to the use of a cell in the manufacture of a medicament for the treatment and/or prevention of a disease

The disease to be treated and/or prevented by the method of the present invention may be immune rejection of the cell which comprises (i) a chimeric antigen receptor (CAR) or a transgenic TCR; and (ii) at least one polypeptide capable of co-localizing an MHC class I polypeptide or MHC class II polypeptide with an intracellular signalling domain within the cell.

The disease may be immune rejection of autologous cells or immune rejection of allogenic cells encoding a CAR or transgenic TCR as described herein.

The MHC class I polypeptide may be HLA-A, HLA-B, HLA-C, HLA-E, HLA-F or HLA-G.

The MHC class II polypeptide may be any HLA-DRα or HLA-DRβ wherein the autologous cell donor has said haplotype.

Both HLA-DP and HLA-DQ have polymorphic alpha and beta chains. Common HLA-DP/DQ alpha or beta chains can be selected for use in the present invention to restrict allogenic production from recipients with that haplotype. The MHC class II polypeptide may be any HLA-DR, DP or DQ wherein the autologous cell donor is homozygous for said haplotype.

The disease to be treated and/or prevented by the methods of the present invention may be an infection, such as a viral infection.

The methods may be for the treatment of a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The CAR cells of the present invention may be capable of killing target cells, such as cancer cells. The target cell may be recognisable by expression of a TAA, for example the expression of a TAA provided above in Table 3.

Depletion of Alloreactive Immune Cells

The methods of the invention may also be for the control of pathogenic immune responses, for example in autoimmune diseases, allergies and graft-vs-host rejection.

The present invention also provides a method for depleting alloreactive immune cells from a population of immune cells, which comprises the step of contacting the population of immune cells with a plurality of cells which express an engineered polypeptide or a bispecific polypeptide as defined herein.

The present invention also provides a method for treating or preventing graft rejection following allotransplantation, which comprises the step of administering a plurality of cells derived from the donor subject to the recipient subject for the allotransplant, wherein the plurality of cells express an engineered polypeptide or a bispecific polypeptide as defined herein.

The present invention also provides a method for treating or preventing graft versus host disease (GVHD) associated with allotransplantation, which comprises the step of contacting the allotransplant with administering a plurality of cells which express an engineered polypeptide or a bispecific polypeptide as defined herein.

The allotransplantation may involve adoptive transfer of allogeneic immune cells.

There is also provided an allotransplant which has been depleted of alloreactive immune cells by a method of the invention. There is also provided an allotransplant which comprises cells of the first aspect of the invention.

There is also provided cells of first aspect of the invention for use in:
depleting alloreactive immune cells from a population of immune cells;
treating or preventing graft rejection following allotransplantation; or
treating or preventing graft versus host disease (GVHD) associated with allotransplantation.

There is also provided the use of cells of first aspect of the invention in the manufacture of a pharmaceutical composition for:
depleting alloreactive immune cells from a population of immune cells;
treating or preventing graft rejection following allotransplantation; or
treating or preventing graft versus host disease (GVHD) associated with allotransplantation.

Method of Making a Cell

CAR or transgenic TCR-expressing cells of the present invention may be generated by introducing DNA or RNA coding for the CAR or TCR and at least one polypeptide capable of co-localizing an MHC class I polypeptide or MHC class II polypeptide with an intracellular signalling domain within the cell within the cell by one of many means including transduction with a viral vector, transfection with DNA or RNA.

The cell of the invention may be made by:
(i) isolation of a cell-containing sample from a subject or one of the other sources listed above; and
(ii) transduction or transfection of the cells with one or more a nucleic acid sequence(s) or nucleic acid construct as defined above in vitro or ex vivo.

The cells may then by purified, for example, selected on the basis of expression of the antigen-binding domain of the antigen-binding polypeptide.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Demonstration of Reduced Allo-Reactivity in a Mixed Lymphocyte Response Assay

Mixed Lymphocyte response (MLR) assays are classical assays which are used to determine allo-reactivity. Normal donor T-cells are transduced with a retroviral vector which expresses a CAR co-expressed with constructs encoding at least one polypeptide capable of co-localizing an MHC class I or II polypeptide with an intracellular signalling domain within the cell. T-cells from the same donor are also transduced with a retroviral vector which just expressed the CAR. These CAR T-cells or CAR/polypeptide capable of co-localizing an MHC class I or II polypeptide with an intracellular signalling domain T-cells are irradiated and repeatedly co-cultured with T-cells from another normal donor who is MHC mismatched. The mismatched T-cells are loaded with tritium which allows counting in response to allo-antigens. After repeated co-culture, the CAR T-cells will have greater allo-responses compared with the CAR/polypeptide capable of co-localizing an MHC class I or II polypeptide with an intracellular signalling domain T-cells.

### Example 2—Demonstration of Reduced Immunogenicity in an Immunocompetent Animal Model

A CAR T-cell cassette is generated which is particularly immunogenic by co-expression of an immunogenic factor such as OVA protein or HSV-TK. In a second CAR T-cell cassette the identical CAR and immunogenic protein are co-expressed along with at least one polypeptide capable of co-localizing an MHC class I or II polypeptide with an intracellular signalling domain. Murine splenocytes are transduced with above constructs. Syngeneic mice are conditioned with low-dose total body irradiation and transduced splenocytes are infused. Engraftment and persistence of CAR T-cells is determined by flow cytometry. Immune responses to the immunogenic factor are determined by ELISPOT. Inclusion of at least one polypeptide capable of co-localizing an MHC class I or II polypeptide with an intracellular signalling domain component is expected to enhance engraftment and reduce immune-responses.

### Example 3—Demonstration of Reduced Allogeneic Response in a Haploidentical Transfer Model (Class I)

BALB/C BLACK6 mice are crossed to result in an F1 hybrid. Engraftment of T-cells from an F1 hybrid mouse would normally result in their rejection after administration to a BalB/C mouse due to recognition of BalB/C MHC molecules. F1 CAR T-cells expressing anti murine CD19, and F1 CAR T-cells expressing both anti-murine CD19 CAR as well as at least one polypeptide capable of co-localizing an MHC class I polypeptide with an intracellular signalling domain are administered to a BalB/C mouse after low-dose total body irradiation. Engraftment is studied serially by bioluminescence imaging and after termination by flow-cytometry.

### Example 4—Demonstration of Reduced Allogeneic Response in a Haploidentical Transfer Model (Class II)

Human/SCID hybrids are generated by engrafting human peripheral blood, human cord blood or human haematopoietic stem cells into NSG mice. After 3-4 weeks, engraftment of human lymphocytes is evident. At this point T-cells from a mismatched human donor are engineered with a CD19 CAR and at least one polypeptide capable of co-localizing an MHC class II polypeptide with an intracellular signalling domain are administered. Control mice receive T-cells from the same donor engineered with just the CD19 CAR. Engraftment of engineered T-cells is studied serially by flow-cytometry (staining for the CD19 CAR) in mouse peripheral blood.

### Example 5—Generation and Testing of Engineered Polypeptides Comprising (i) CD79a Fused to CD3z Endodomain and CD79a Fused to CD3z Endodomain; and (ii) MHC Class II DRα Extracellular Domain Fused to CD3z Endodomain

(i) MHC II has been shown to interact with the CD79α/CD79b and that engagement of MHC II molecules by TCRs results in the phosphorylation of tyrosine residues present in the intracellular domains of CD79α/b and the initiation of a signal transduction cascade.

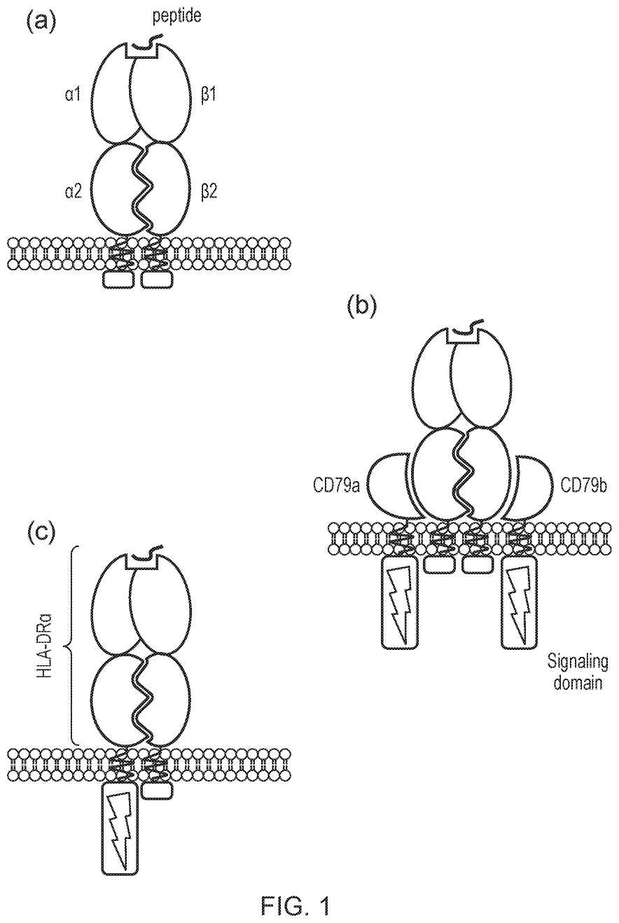

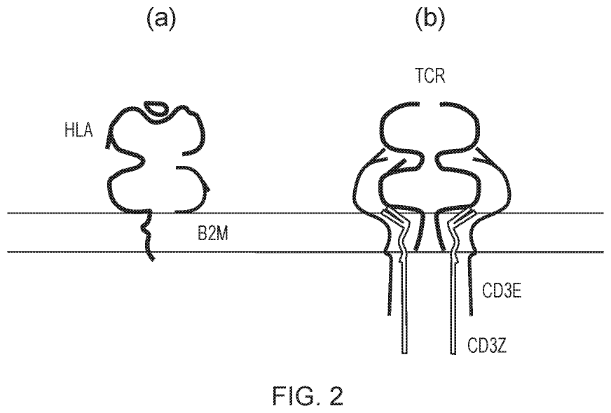

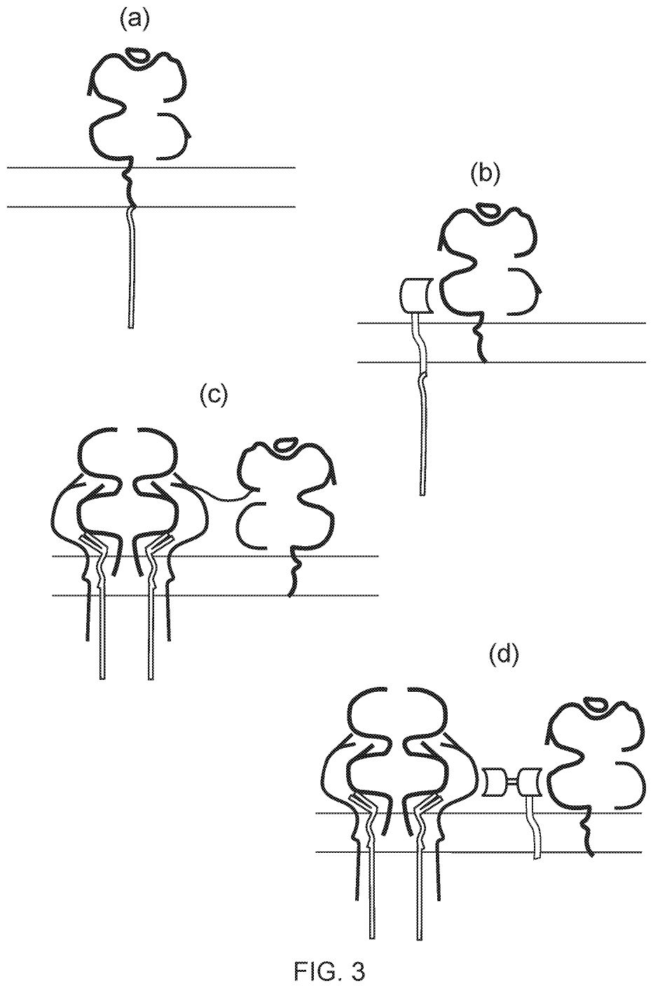

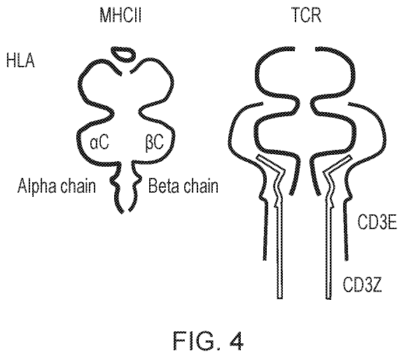

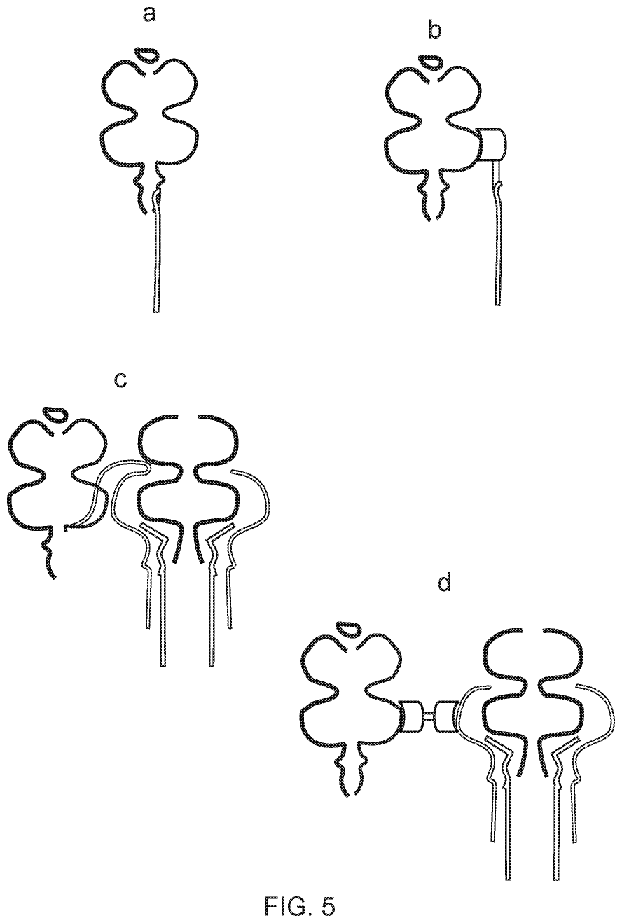

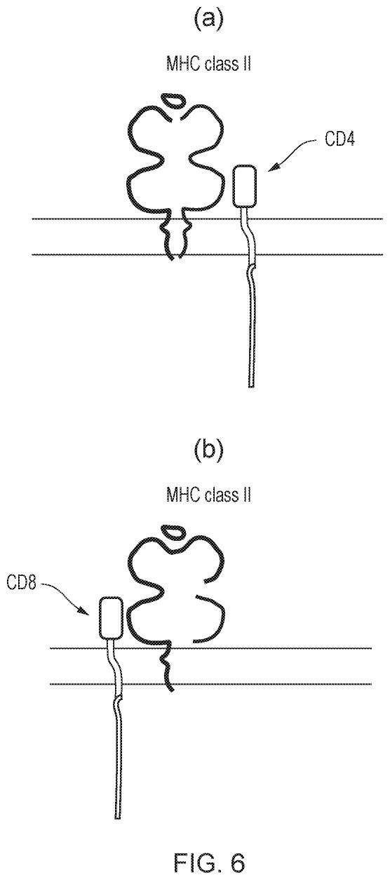

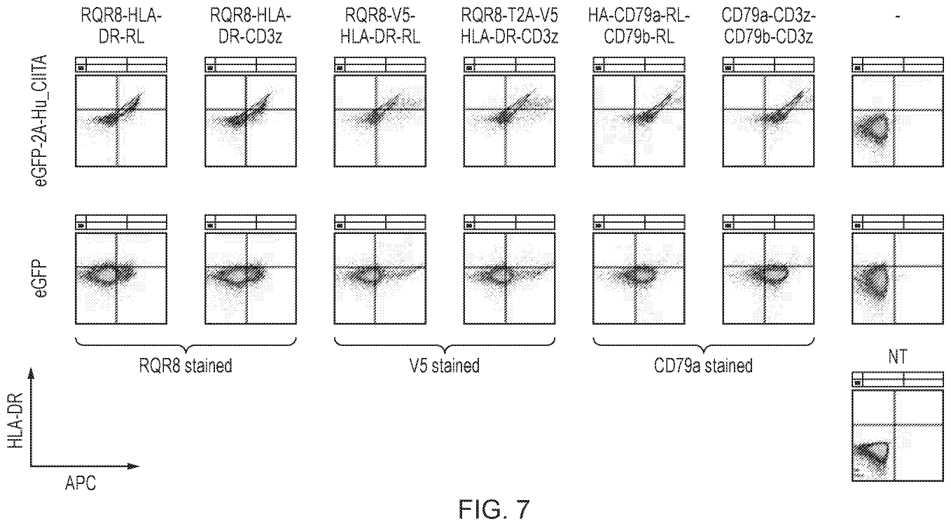

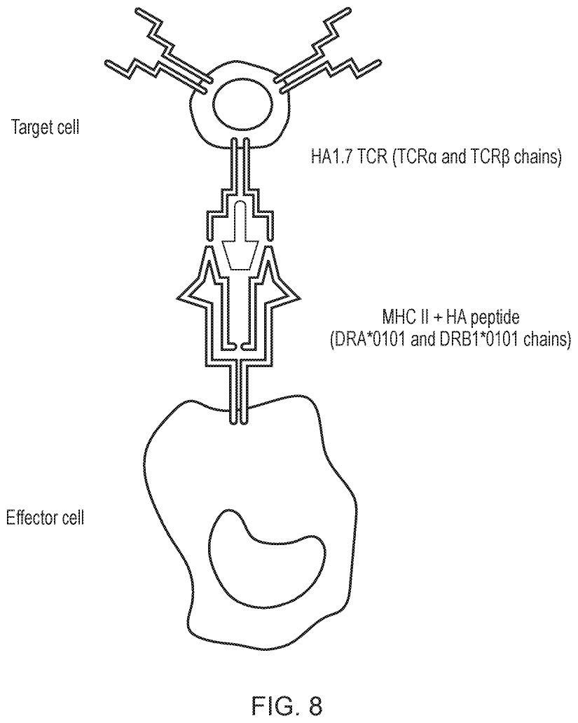

To determine whether the association between MHC II and CD79α/b can be used to transduce an activation signal to the T-cell, fusions between the extracellular and transmembrane domains of CD79α/b and the CD3z chain were made as shown in Table 4. A CD79α/b and CD3 chimeras, as produced by the last plasmid in the table, are illustrated schematically in FIG. 1(b).

TABLE 4
CD79a/b and CD3z chimeric constructs.
Plasmid

```
    SFGmR.HA-T2A_CD79a-Tm-full_P2A_CD79b-Tm-full
    SFGmR.HA-T2A-CD79a-linker-RL-P2A-CD79b-linker-RL
    SFGmR.HA-T2A-CD79a-linker-CD3z-P2A-CD79b-linker-CD3z
```

HA—hemagglutinin marker

T2A/P2A—2A peptide cleavage site

TM—transmembrane domain

RL—Rigid linker

(ii) MHC II molecules are expressed on the surface of active, but not resting T primary cells. Similarly, the Jurkat cell line (T-cell lymphoblastic) does not express MHC II in the absence of activation, but its expression is induced after stimulation through the T-cell receptor.

The second approach to engineering chimeric antigen receptor T cells capable of eliminating autoreactive T cells, involved generating a fusion between the MHC class II DRα extracellular and transmembrane domain and the intracellular signalling domain of CD3ζ (Table 5). A DRα and CD3 chimeric receptor is illustrated schematically in FIG. 1c.

TABLE 5
DRα and CD3ζ chimeric constructs
Plasmid

```
    SFGmR.RQR8-T2A-HLA_DRalpha-linker-RL
    SFGmR.RQR8-T2A-HLA_DRalpha-linker-CD3z 30
    SFGmR.RQR8-T2A-V5-HLA_DRalpha-linker-RL
    SFGmR.RQR8-T2A-V5-HLA_DRalpha-linker-CD3z
```

V5 is a marker, detectable using antibodies to the V5 epitope.

The constructs are tested by transducing Jurkat cells with the plasmids and stimulating the cells with anti-DRα antibodies induces crosslinking of the MHC II molecules. Activation of the cells is monitored by staining the cells with anti-CD69 antibody, which is an early activation marker and is upregulated within 24 hours of stimulation.

This approach relies on the cell surface expression of MHC II. The transcription factor CIITA regulates MHC II and forced over expression of CIITA induces cell surface expression of MHC II. To facilitate testing of the CD79a/b and DRα constructs, Jurkat cells were transduced with a construct expressing CIITA and eGFP (transduction marker) and the constructs described in Table 2 above.

These Jurkat cells were stained with anti-DRα antibody and antibodies to transduction markers (anti-CD34 for RQR8 or anti-V5 tag or anti-CD79) and analysed by flow cytometry to verify that there was cell surface expression of MHC II and the chimeras under study (FIG. 7). The results indicated that transduction of Jurkat cells with CIITA induces cell surface expression of DRα.

### Example 6—Cytotoxicity Assays for Cell Expressing Engineered Polypeptide Comprising MHC Class II Fused to CD3z Endodomain

Cytotoxicity assays are set up using an α/β T-cell receptor (TCR), HA1.7, known to recognise the immunodominant peptide derived from hemagglutinin of influenza virus A. The immunodominant peptide (PKYVKQNTLKLAT) is DR1-restricted and can be presented by either DRA*0101 and DRB1*0101 or DRA*0101 and DRB1*0401 MHC II molecules and recognised by the HA1.7 α/βTCR. Target cells are CD4+ cell lines transduced to express the HA1.7 α/βTCR, while effector cells are cytotoxic T-cells (CD8+) transduced to express DRA*0101 and DRB1*0101 or DRA*0101 MHC II chimeras fused to the signalling domains of CD3ζ and the HA immunodominant peptide (FIG. 8). Challenge of the MHC II/CD3z cytotoxic T-cells with the HA1.7 α/βTCR+ cells should result in elimination of the target cells and this is assessed by analysis of the cell populations by flow cytometry.

### Example 7—Generation and Testing of Engineered Polypeptide Comprising CD4 Variable Domain Fused to CD3z Endodomain

PBMCs are transduced to express a the marker gene RQR8 described in WO2013/153391 and a fusion protein consisting of WT or mutant high affinity form of CD4 variable domain (26-125aa Uniprot P01730) tethered to CD3zeta via a flexible linker (CD4-CD3). For this assay, target PBMCs from the same donor are co-cultured with effector cells in the presence or absence of superantigens (SAgs) to ligate the armed MHC to the TCR. Superantigens are not processed intracellularly. Instead, they bind class II MHC molecules as intact macromolecules and bind outside of the peptide-antigen binding groove. SAgs are molecules that indiscriminately stimulate up to 20% of all T cells (normal response to antigen stimulates only 0.01% of T cells).

Live transduced T cells are enumerated after 72 h of co-culture and each condition normalized to its respective non-transduced co-culture.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which comprises:
(i) a chimeric antigen receptor (CAR) or a transgenic T-cell receptor (TCR); and
(ii) at least one polypeptide capable of co-localizing an MHC class I polypeptide or an MHC class II polypeptide with an intracellular signalling domain within the cell.

2-4. (canceled)

5. A cell according to claim 1, which comprises:
(i) a chimeric antigen receptor (CAR) or a transgenic T-cell receptor (TCR); and
(ii) an engineered polypeptide which comprises a binding domain which is binds to an MHC class I polypeptide or an MHC class II polypeptide linked to an intracellular signalling domain.

6. A cell according to claim 1, which comprises:
(i) a chimeric antigen receptor (CAR) or a transgenic T-cell receptor (TCR); and
(ii) an engineered polypeptide which comprises CD79α or CD79β linked to an intracellular signalling domain.

7. A cell according to claim 1, which comprises:
(i) a chimeric antigen receptor (CAR) or a transgenic T-cell receptor (TCR); and
(ii) an engineered polypeptide which comprises the MHC class II-binding domain of CD4 linked to an intracellular signalling domain.

8. A cell according to claim 7, wherein the MHC class II-binding domain of CD4 comprises one or more mutations to increase its binding affinity for the β2 region of MHC class II.

9. A cell according to claim 8, wherein the MHC class II-binding domain of CD4 comprises substitution mutations Gln40Tyr and/or Thr45Trp with reference to SEQ ID No. 47.

10. A cell according to claim 1, which comprises:
(i) a chimeric antigen receptor (CAR) or a transgenic T-cell receptor (TCR); and
(ii) an engineered polypeptide which comprises the MHC class I-binding domain of CD8 linked to an intracellular signalling domain.

11. A cell according to claim 1, which comprises:
(i) a chimeric antigen receptor (CAR) or a transgenic T-cell receptor (TCR); and expresses
(ii) a bispecific polypeptide which comprises;
(a) a first binding domain which is binds to an MHC class I polypeptide or an MHC class II polypeptide; and
(b) a second binding domain which binds to a polypeptide comprising an intracellular signalling domain or a component of the CD3 complex.

12. A cell according to claim 11, wherein the bispecific polypeptide is membrane-tethered.

13. A nucleic acid construct which comprises:
(i) a first nucleic acid sequence which encodes a chimeric antigen receptor (CAR) or a transgenic TCR; and
(ii) a second nucleic acid sequence which encodes an engineered polypeptide which, when expressed in a cell, is capable of co-localizing an MHC class I polypeptide or an MHC class II polypeptide with an intracellular signaling domain within the cell.

14. A nucleic acid construct according to claim 13 wherein the first and second nucleic acid sequences are separated by a co-expression site.

15. (canceled)

16. A vector which comprises a nucleic acid construct according to claim 13.

17. (canceled)

18. A pharmaceutical composition which comprises a plurality of cells according to claim 1.

19. (canceled)

20. A method for treating a disease, which comprises the step of administering a pharmaceutical composition according to claim 18 to a subject.

21-22. (canceled)

23. A method for making a cell according to claim 1, which comprises the step of introducing:
(i) a first nucleic acid sequence which encodes a chimeric antigen receptor (CAR) or a transgenic TCR; and
(ii) a second nucleic acid sequence which encodes an engineered polypeptide which, when expressed in a cell, is capable of co-localizing an MHC class I polypeptide or an MHC class II polypeptide with an intracellular signalling domain within the cell, into a cell ex vivo.

24. A method for depleting alloreactive immune cells from a population of immune cells, which comprises the step of contacting the population of immune cells with a plurality of cells which express an engineered polypeptide capable of co-localizing an MHC class I polypeptide or an MHC class II polypeptide with an intracellular signaling domain within the cell.

25-28. (canceled)


SEQUENCE LISTING (61 sequences)

SEQ ID NO: 1 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 2 - protein, 362 aa, Homo sapiens [sequence omitted]

SEQ ID NO: 3 - protein, 257 aa, Homo sapiens [sequence omitted]

SEQ ID NO: 4 - protein, 249 aa, Homo sapiens
SWKKALRIPGDLRVATVTLMLAMLSSLLAEGRDSPEDFVYQFKGLCYFTNGTERVRLVTRYIYNREEYARFDSDVGVYRAVTPQGRPVAEYWNSQKEVLERTRAELDTVCRHNYEVGYRGILQRRVEPTVTISPSRTEALNHHNLLVCSVTDFYPGQIKVQWFRNDQEETAGVVSTPLIRNGDWTFQILMLEMTPQRGDVYTCHVEHPSLQSPITVEWRAQSESAQSKMLSGVGGFVLGLIFLGLGLII

SEQ ID NO: 5 - protein, 23 aa, Artificial Sequence
  CD8 alpha transmembrane domain
IYIWAPLAGTCGVLLLSLVITLY

SEQ ID NO: 6 - protein, 28 aa, Artificial Sequence
  CD28 alpha transmembrane domain
FWVLVVVGGVLACYSLLVTVAFIIFWVR

SEQ ID NO: 7 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 8 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 9 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 10 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 11 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 12 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 13 - protein, 5 aa, Artificial Sequence
  linker peptide motif
SGGGG

SEQ ID NO: 14 - protein, 16 aa, Artificial Sequence
  linker peptide
SGGGGSGGGGSGGGGS

SEQ ID NO: 15 - protein, 347 aa, Artificial Sequence
  polypeptide comprising an ectodomain from HLA-DRalpha, a transmembrane domain and an intracellular CD3-zeta endodomain
METDTLLLWVLLLWVPGSTGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 16 - protein, 389 aa, Artificial Sequence
  polypeptide comprising an ectodomain from HLA-DRalpha, a transmembrane domain, a 41BB endodomain and an intracellular CD3-zeta endodomain
METDTLLLWVLLLWVPGSTGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 17 - protein, 388 aa, Artificial Sequence
  polypeptide comprising an ectodomain from HLA-DRalpha, a transmembrane domain, a CD28 endodomain and an intracellular CD3-zeta endodomain
METDTLLLWVLLLWVPGSTGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 18 - protein, 256 aa, Artificial Sequence
  polypeptide comprising an ectodomain from CD79alpha, a 41BB domain and an endodomain from CD79
METDTLLLWVLLLWVPGSTGLWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELRKRWQNEKLGLDAGDEYEDENLYEGLNLDDCSMYEDISRGLQGTYQDVGSLNIGDVQLEKP

SEQ ID NO: 19 - protein, 262 aa, Artificial Sequence
  polypeptide comprising an ectodomain from CD79beta, a CD28 domain and an endodomain from CD79
METDTLLLWVLLLWVPGSTGARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRSKRSRLLHSDYMNMTPRRPGPTRKHYQPYAPPRDFAAYRSLDKDDSKAGMEEDHTYEGLDIDQTATYEDIVTLRTGEVKWSVGEHPGQE

SEQ ID NO: 20 - protein, 255 aa, Artificial Sequence
  polypeptide comprising an ectodomain from CD79alpha, a CD28 domain and an endodomain from CD79
METDTLLLWVLLLWVPGSTGLWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSRKRWQNEKLGLDAGDEYEDENLYEGLNLDDCSMYEDISRGLQGTYQDVGSLNIGDVQLEKP

SEQ ID NO: 21 - protein, 263 aa, Artificial Sequence
  polypeptide comprising an ectodomain from CD79beta, a 41BB domain and an endodomain from CD79
METDTLLLWVLLLWVPGSTGARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLKRGRKKLLYIFKQPFMRPVQTTQEEDGCSCRFPEEEEGGCELLDKDDSKAGMEEDHTYEGLDIDQTATYEDIVTLRTGEVKWSVGEHPGQE

SEQ ID NO: 22 - protein, 308 aa, Artificial Sequence
  polypeptide comprising an ectodomain from CD79alpha, a 41BB domain and a CD3-zeta domain
METDTLLLWVLLLWVPGSTGLWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 23 - protein, 328 aa, Artificial Sequence
  polypeptide comprising an ectodomain from CD79beta, a 41BB domain and a CD3-zeta domain.
METDTLLLWVLLLWVPGSTGARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDIITAEGIILLFCAVVPGTLLLFKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 24 - protein, 112 aa, Artificial Sequence
  CD3-zeta endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 368 aa, Artificial Sequence
  4-1BB and CD3-zeta endodomains
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - protein, 152 aa, Artificial Sequence
  CD28 and CD3-zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 188 aa, Artificial Sequence
  CD28, OX40 and CD3-zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 365 aa, Artificial Sequence
  HLA-A01, illustrative amino acid sequence of HLA class I - HLA-A
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 29 - protein, 365 aa, Artificial Sequence [sequence omitted]

SEQ ID NO: 30 - protein, 365 aa, Artificial Sequence
  HLA-A03, illustrative amino acid sequence of HLA class I - HLA-A
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 31 - protein, 362 aa, Artificial Sequence
  HLA-B07, illustrative amino acid sequence of HLA class I - HLA-B
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 32 - protein, 362 aa, Artificial Sequence
  HLA-B08, illustrative amino acid sequence of HLA class I - HLA-B
MLVMAPRTVLLLLSAALALTETWAGSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 33 - protein, 362 aa, Artificial Sequence
  HLA-B44, illustrative amino acid sequence of HLA class I - HLA-B
MRVTAPRTLLLLLWGAVALTETWAGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 34 - protein, 366 aa, Artificial Sequence
  HLA-C01, illustrative amino acid sequence of HLA class I - HLA-C
MRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 35 - protein, 441 aa, Artificial Sequence
  HLA-A01-CAR polypeptide
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 36 - protein, 441 aa, Artificial Sequence
  HLA-A02-CAR polypeptide
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 37 - protein, 441 aa, Artificial Sequence
  HLA-A03-CAR polypeptide
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 38 - protein, 441 aa, Artificial Sequence
  HLA-B07-CAR polypeptide
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 39 - protein, 441 aa, Artificial Sequence
  HLA-B08-CAR polypeptide
MLVMAPRTVLLLLSAALALTETWAGSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 441 aa, Artificial Sequence
  HLA-B44-CAR polypeptide
MRVTAPRTLLLLLWGAVALTETWAGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 41 - protein, 442 aa, Artificial Sequence
  HLA-C01-CAR polypeptide
MRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSSQPTIPIVGIVAGLAVLAVLAVLGAVVAVVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - protein, 4 aa, Artificial Sequence
  ITAM (immunoreceptor tyrosine-based activation motif)
  misc_feature (2)..(3): Xaa can be any naturally occurring amino acid
  MISC_FEATURE (4)..(4): Xaa may be Leu or Ile
YXXX

SEQ ID NO: 43 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 44 - protein, 7 aa, Artificial Sequence
  consensus Tobacco Etch Virus (TEV) cleavage site
ENLYFQS

SEQ ID NO: 45 - protein, 7 aa, Artificial Sequence
  linker sequence
SGGGSDP

SEQ ID NO: 46 - protein, 6 aa, Artificial Sequence
  serine-glycine linker
SGGGGS

SEQ ID NO: 47 - protein, 100 aa, Artificial Sequence
  CD4 D1 domain
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGL

SEQ ID NO: 48 - protein, 114 aa, Artificial Sequence
  CD8alpha Ig-like V-type domain
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPA

SEQ ID NO: 49 - protein, 242 aa, Artificial Sequence
  single-chain variable fragment (scFv) sequence comprising CDR sequences from OKT3
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 50 - protein, 13 aa, Artificial Sequence
  OKT3 heavy chain complementarity determining region (CDR) CDR1
KASGYTFTRYTMH

SEQ ID NO: 51 - protein, 16 aa, Artificial Sequence
  OKT3 heavy chain CDR2
INPSRGYTNYNQKFKD

SEQ ID NO: 52 - protein, 10 aa, Artificial Sequence
  OKT3 heavy chain CDR3
YYDDHYCLDY

SEQ ID NO: 53 - protein, 10 aa, Artificial Sequence
  OKT3 light chain CDR1
SASSSVSYMN

SEQ ID NO: 54 - protein, 11 aa, Artificial Sequence
  OKT3 light chain CDR2
RWIYDTSKLAS

SEQ ID NO: 55 - protein, 9 aa, Artificial Sequence
  OKT3 light chain CDR3
QQWSSNPFT

SEQ ID NO: 56 - protein, 244 aa, Artificial Sequence
  scFv sequence comprising CDR sequences from OKT3
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRSSSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 57 - protein, 26 aa, Artificial Sequence
  IgG1 hinge linker sequence
AEPKSPDKTHTCPPCPKDPKSGGGGS

SEQ ID NO: 58 - protein, 45 aa, Artificial Sequence
  CD8 stalk linker sequence
TTTPAPRPPTPAPTIASQPLSLRPEACRPAAGGAVHTRGLDFACD

SEQ ID NO: 59 - protein, 5 aa, Artificial Sequence
  peptide spacer repeating unit
GGGGS

SEQ ID NO: 60 - protein, 248 aa, Artificial Sequence
  CD8alphaalpha homodimer
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAGGGGSGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPA

SEQ ID NO: 61 - protein, 111 aa, Artificial Sequence
  CD8beta Ig-like V-type domain
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQL